(19) United States
(12) Patent Application Publication
(10) Pub. No.: US 2026/0355384 A1
(43) Pub. Date: Nov. 12, 2026

(54) HEMOSTATIC MATERIALS, METHODS OF MANUFACTURE, AND USES THEREOF

(71) Applicants: BAXTER INTERNATIONAL INC., Deerfield, IL (US); BAXTER HEALTHCARE SA, Glattpark (Opfikon) (CH)

(72) Inventors: Raffael REICHSÖLLNER, Vienna (AT); Tina GURMANN, Vienna (AT); Paul SLEZAK, Vienna (AT); Heinz REDL, Vienna (AT); Robert LISKA, Wien (AT); Stefan BAUDIS, Vienna (AT); Heinz GULLE, Gross Enzersdorf (AT)

(21) Appl. No.: 19/559,230

(22) Filed: Mar. 6, 2026

Related U.S. Application Data
(60) Provisional application No. 63/800,079, filed on May 5, 2025.

(51) Int. Cl. A61L 26/00

(52) U.S. Cl. CPC A61L 26/0014; A61L 26/0066; A61L 26/009; A61L 2400/04

(57) ABSTRACT

A hemostatic product is suitable for the administration to a human patient and includes a biocompatible hemostatic material containing non-porous, cross-linked polyvinyl alcohol particles (PVA particles). Methods for the production of this product and uses thereof are also disclosed, including a method for treatment of a wound or for achieving hemostasis in a human patient, by administering the hemostatic product to a patient with a wound or in need of hemostasis.

### CROSS REFERENCE TO RELATED APPLICATIONS

This application claims priority to U.S. Provisional Application No. 63/800,079 filed May 5, 2025, the entire contents of which are incorporated herein by reference.

### FIELD OF THE INVENTION

The present specification relates to biocompatible hemostatic materials and the production and use thereof.

Wounds are generally classified as acute or chronic in accordance with their healing tendencies. Acute wounds, typically those occurring as a result of surgery or trauma, usually heal uneventfully within an expected time frame. Acute wounds include wounds such as active bleeding wound sites, e.g., wounds that have detectable, un-clotted blood. The rapid control of topical bleeding at active bleeding wound sites is of critical importance in wound management, especially for the management of trauma, e.g., as a result of traumatic injury or surgery. Indeed, uncontrolled bleeding is among the leading causes of mortality in traumatic and surgical injuries. Conventional methods of controlling bleeding at active bleeding sites employ the use of “passive” devices including cotton gauze pads. Cotton pads, however, do not have the ability to initiate or accelerate blood clotting.

In contrast to passive devices, hemostats are substances that promote hemostasis (stop bleeding). A wide variety of hemostats are available to improve hemostasis during surgical procedures. Some are considered active agents, because they contain, for example, fibrinogen or thrombin, and actively participate in the coagulation cascade to form a fibrin clot.

The idea of promoting hemostasis using fibrin was first reported by Bergel in 1909 (Bergel, Dt. Med. Wschr. 35 (1909), 663-665), and fibrin sealants have been commercially available in Europe and Japan for over 40 years with a good safety profile. However, it was not until 1998 that the US Food and Drug Administration (FDA) approved their use due to concerns over the transmission of infectious disease, as these products were developed from pooled plasma components.

Thrombin is a serine protease that plays important roles in blood clotting (coagulation). As the key coagulation protease, thrombin converts soluble fibrinogen into fibrin networks crosslinked by a transglutaminase (FXIII). In addition, thrombin is the most potent activator of platelets by stimulating protease-activated receptors (PAR). Upon activation by thrombin, platelets physically alter the conformation of GP IIb/IIIa receptors and provide high-affinity binding sites for fibrinogen, providing fibrinogen-crosslinked platelet aggregation. It has been proven that the short TRAP6 peptide (SFLLRN) can work as a potent platelet activator separately and stimulates platelet aggregation via PAR-1 signaling. Other TRAPs which have been disclosed which activate fibroblasts and are implicated in wound healing include peptides TRAP 508-530, amino acids AGYKPDEGKRGDACEGDSGGPFV; and TRAP 517-530, amino acids RGDACEGDSGGPFV.

However, commercial hemostats formulated with biologically-derived materials, including high thrombin concentrations, can lead to adverse effects including apoptosis of human keratinocytes. For example, WO 1996/40033 A1 discloses a material with hemostatic agents, including epsilon aminocaproic acid and a TRAP, wherein the hemostatic agents are sprayed or coated on a hemostatic matrix to obtain a matrix wherein the agent is physically (but not covalently) adsorbed on the matrix. Such patches have the drawback that the hemostatic agents provided therewith easily release from the patch when contacted to a bleeding area of a patient. As such, there is the potential danger of inducing systemic thrombotic events. Further, current hemostatic powders often contain animal- and/or human-derived components such as gelatin, thrombin, fibrinogen, or collagen which may induce unwanted immunological responses or carry infectious risks, especially due to viral contamination or for transmitting transmissible spongiform encephalopathies (TSEs; a group of fatal neurodegenerative diseases that affect humans and animals caused by a transmissible agent called a prion; TSEs include classical and atypical bovine spongiform encephalopathy (BSE) in cattle.

Thus, all these treatments suffer from inconsistent effectiveness and the risk of unwanted side effects; therefore, improved systems, devices, and methods are desirable. Using recombinant components may provide reduced risk concerning viral contamination and batch-to-batch reproducibility but are not free of such risks and bear the possibility of reduced activity compared to their natural counterparts or—due to or depending on the production process—the possibility of introducing neo-antigens.

Moreover, production of hemostats with biologically-derived material is also more complicated compared to production of synthetic materials. Batches of biological substance can vary in their composition, activity, source, etc. which provides a burden for mass production, especially with respect to reproducibility with respect to effectiveness and quality control. However, fully synthetic hemostats have not yet proven to be sufficiently effective for the control of wounds in human medicine, especially when urgent hemostatic activity is needed, e.g. in surgery or emergency wound treatment.

It is an object of the present invention to provide a hemostatic material which is not inherently connected and loaded with such risks, especially with the risk of viral contamination. It is a further object to provide efficient hemostats which can be easily produced by mass production by reproducible methods and without the risks and downsides of material which is obtained from biological sources, especially of human or non-human mammalian sources, or from recombinant production systems.

### SUMMARY

Therefore, the present invention provides a hemostatic product suitable for the administration to a human patient comprising a biocompatible hemostatic material comprising or consisting of non-porous, cross-linked polyvinyl alcohol particles (PVA particles). The material according to the present invention may be used alone or in the form of a kit, further comprising at least one administration device, buffer solution, syringe, tube, catheter, forceps, scissors, sterilizing pad or lotion; in surgery or in the treatment of injuries or wounds.

The present invention also provides a method for producing a hemostatic product comprising cross-linked non-porous PVA particles, wherein polyvinyl alcohol is modified and/or cross-linked, and then optionally washed and dried.

The present invention also provides a method for treating wounds or achieving hemostasis by administering the hemostatic product according to the present invention to a patient.

### BRIEF DESCRIPTION OF THE DRAWINGS

FIG. 1 shows the schematic illustration steps involved in the production of PVA-NB derived hemostatic particle matrix prototypes: A) In a first step the linear polymer poly (vinyl alcohol) is modified with pendant norbornene groups by reaction with carbic anhydride. The resulting PVA-NB macromer is soluble in water. B) Solutions of PVA-NB, a suitable multifunctional thiol as crosslinker and a photoinitiator (“PI”) are irradiated with a UV light source to form crosslinked hydrogels. C) Hydrogels are dried to remove the solvent to obtain the dry network matrix for particle production via milling.

FIG. 2 shows the UV-induced crosslinking of norbornene-modified PVA (PVA-NB) with Glycol di(3-mercapto acetate) as dithiol crosslinker.

FIG. 3 shows SEM images of pre-(left) and post-sterilized (right) crosslinked PVA-particles (PVA-NB7.5-GDMA50). Samples were sputter coated prior to the picture taking process. Magnifications: 22×, 200×, 600× and 1000×. White dotted lines: Magnified area on the following image. Pictures were taken in secondary electron mode and high vacuum. Yellow arrows: consequences of milling process, marks originate from impact of mill blades.

FIG. 4 shows the synthesis of PVA from vinyl acetate and tautomerization of vinyl alcohol to acetaldehyde making direct polymerization of vinyl alcohol impossible as route to PVA (in the further schematic drawings, the acetyl groups are omitted for clarity).

FIG. 5 shows the modification of PVA with methacrylate groups using glycidyl methacrylate or methacrylic anhydride.

FIG. 6 shows the synthetic pathways towards PVA-NB and PVA-Norb. For PVA-NB, reaction of carbic anhydride with a nucleophile like the OH-groups on the PVA backbone results in a ring-opening reaction leading to a free carboxylic acid that can be neutralized with bicarbonate to yield the corresponding sodium salt form. For PVA-Norb, carbodiimide-coupling of two equivalents of 5-norbornene-2-carboxylic acid yields the corresponding carboxylic anhydride that can then be used to yield a non-ionic variant of PVA-norbornene.

FIG. 7 shows the crosslinking of PVA-NB macromer with DTT as bifunctional thiol via a radical-mediated thiol-ene step-growth reaction. Radicals are generated due to UV irradiation of the aqueous hydrogel precursor formulation containing a photoinitiator (Irgacure 2959, 12959, α-hydroxy-4-(2-hydroxyethoxy)-α-methylpropiophenon). Source of figure: Qin et al., Adv. Funct. Mat. 25 (2015), 6606-6617.

FIG. 8 shows the volumetric swelling of pre- and post-sterilized PVA-particles (PVA-NB7.5-GDMA50). Swelling was conducted in PBS for a total time of 48 h (0-60 min: n=3, 1440-2880 min: n=2).

FIG. 9 shows the statistical analysis of volumetric swelling results of pre- and post-sterilized PVA-particles (PVA-NB7.5-GDMA50) after 60 minutes (n=3). One-way ANOVA (significance level=0.05) and Tukey test were performed.

FIG. 10 shows water uptake of pre- and post-sterilized PVA-particles (PVA-NB7.5-GDMA50). Swelling was conducted in PBS for 24 h at room temperature.

FIG. 11 shows the results of the in vitro degradability study on pre- and post-sterilized PVA particles (PVA-NB7.5-GDMA50). Mass in percent (%) of the initial weight of the samples after 7, 14, 21, 28 and 35 days of storage in PBS (pH=7.4) at 37 °C. Tests were performed in triplicate (grey: individual measurements). Mean values are depicted in black.

FIG. 12 shows cell viability (%) of MG63 cells in presence of 10 mg/mL pre- and post-sterilized crosslinked PVA particles (PVA-NB7.5-GDMA50). Cell viability was assessed with the help of a direct BrdU ELISA assay (n=16).

FIG. 13 shows the water uptake of particles for different types of PVA modification. Unmodified PVA was tested as a reference group, and PVA-NB and PVA-Norb for comparison of ionic and non-ionic norbornene variants (i.e. “PVA (unmod)”, “PVA-NB7-DTT100”, “PVA-Norb5-DTT100”). Swelling was conducted in PBS for 24 h at room temperature.

FIG. 14 shows the relative volume increase of the particle phase during swelling brought on by contact with PBS for particles based on different PVA modification variants (i.e. “PVA (unmod)”, “PVA-NB7-DTT100”, “PVA-Norb5-DTT100”). Swelling was conducted in PBS for 48 h at room temperature.

FIG. 15 shows the water uptake of materials (“PVA-NB7-DTT100”) for different size fractions of particles as indicated. Swelling was conducted in PBS for 24 h at room temperature.

FIG. 16 shows the relative volume increase of the particle phase during swelling brought on by contact with PBS for different size fractions of PVA particles (“PVA-NB7-DTT100”). Swelling was conducted in PBS for 48 h at room temperature.

FIG. 17 shows the chemical structures of DTT, thiolated PEG, and mercaptoacetic acid- and mercaptopropionic acid-derived multifunctional thiols with their abbreviated names.

FIG. 18 shows the schematic reaction of ester hydrolysis for network degradation. Schematic chemical structures of crosslinks formed between PVA-NB macromers with either DTT or GDMA crosslinks. Blue lines mark ester bonds for potential network-degrading hydrolysis.

FIG. 19 shows the water uptake of materials to investigate variations of DS of PVA-NB and molar content of crosslinker amount (i.e. “PVA (unmod)”, “PVA-NB7-DTT50”, “PVA-NB7-DTT100”, PVA-NB14-DTT50″). Swelling was conducted in PBS for 24 h at room temperature.

FIG. 20 shows the relative volume increase measurements to investigate variations of DS of PVA-NB and molar content of crosslinker amount (i.e. “PVA (unmod)”, “PVA-NB7-DTT50”, “PVA-NB7-DTT100”, PVA-NB14-DTT50″). Swelling was conducted in PBS for 48 h at room temperature.

FIG. 21 shows the chemical structures of bifunctional thiols DTT and GDMA used as crosslinkers for radical-mediated thiol-ene network formation with PVA-NB as ene-containing macromer.

FIG. 22 shows the water uptake measurements to investigate the influence of variation of crosslinker type and concentration. DTT and GDMA were used as model thiols in varying relative molar equivalents (i.e. “PVA (unmod)”, “PVA-NB7-DTT50”, “PVA-NB7-DTT100”, “PVA-NB7-GDMA50”, “PVA-NB7-GDMA100”). Swelling was conducted in PBS for 48 h at room temperature.

FIG. 23 shows the measurements of relative volume increases to investigate the influence of crosslinker type and concentration variation. DTT and GDMA were used as model thiols in varying relative molar equivalents (i.e. “PVA (unmod)”, “PVA-NB7-DTT50”, “PVA-NB7-DTT100”, “PVA-NB7-GDMA50”, “PVA-NB7-GDMA100”). Swelling was conducted in PBS for 48 h at room temperature.

FIG. 24 shows small (<200 μm), large (200-500 μm) and mixed (<500 μm) particle fractions.

FIG. 25 shows application of large particles after 2 minutes (A), after 3 minutes, oozing bleeding (B) and after 8 minutes, hemostasis achieved (C).

FIG. 26 shows application of small particles after 3 minutes (A), after 5 minutes, oozing bleeding (B) and after 8 minutes, hemostasis achieved (C).

FIG. 27 shows application of mixed particles after 2 minutes, hemostasis achieved (A), after 3 minutes (B).

FIG. 28 shows application as slurry with thrombin after 2 minutes (A), after 3 minutes (B).

FIG. 29 shows the experimental setup to quantify particle swelling. PBS is added to dry particles at the start of the experiment and the particle phase volume is noted throughout the experiment as a function of time on the graduation markings of the syringe barrel (indicated by orange arrows).

FIG. 30 shows Nano CT measurements for PVA-NB7-DTT172 oven-dried particles according to the present invention (A), PVA-NB7-DTT172 lyophilized particles (B), PF 1 27 kDa foamed particles (C) and PF 2 27 kDa freeze/thawed particles (D).

FIG. 31 shows Nano CT evaluation for PVA-NB7-DTT172 oven-dried particles according to the present invention, PVA-NB7-DTT172 lyophilized particles, PF 1 27 kDa foamed particles and PF 2 27 kDa freeze/thawed particles.

### DETAILED DESCRIPTION

The present invention discloses a hemostatic product with commercial suitability as a passive particle hemostat. The PVA particles according to the present invention are crosslinked and of non-porous nature. The particles are biocompatible and biodegradable which means that the particles do not threaten, poison, impede, or adversely affect living tissue (e.g. human tissue that is contacted with the material) and that the particles are naturally absorbed by the patient's body after some time, usually between a few days and a few months. For example, more than 90% of the PVA particles according to the present invention are absorbed by a human patient's body within 6 months after administration. Usually, the absorption is even much quicker so that more than 90% of the particles are absorbed within 3 months, preferably within 2 months, especially within 1 month after administration to a human patient.

In contrast to porous, sponge-structured PVA hemostats (as e.g. disclosed in WO 03/063922 A1), the hemostatic PVA particles according to the present invention do not rely on the pore structure with respect to swelling and fluid uptake but on the physicochemical nature of the particles which are able to keep fluid in the material (and not in pores inside the material). This has also been shown in the course of the present invention i.a. by the comparative tests as disclosed in Example 12 in the example section below, wherein the non-porous nature of the PVA particles is evidenced i.a. by Nano-CT analysis.

WO 2017/037178 A1 discloses a biocompatible matrix, preferably a PVA matrix, wherein a thrombin receptor activating agent is covalently coupled to this matrix to form a hemostatic compound. Surprisingly, it turned out in the course of the present invention that the carrier material used in the WO 2017/037178 A1 (as an intermediate product) was itself a very suitable hemostat (as a final product engineered and finalized for human use), even without the thrombin receptor activating agent covalently coupled thereto so that a novel hemostatic product can be established on the PVA matrix only without the need to add or combine further hemostatic products of natural or recombinant origin, such as blood products.

The hemostatic product according to the present invention can therefore be supplied as a “fully synthetic” hemostat which does not require addition of materials from risky sources, such as material from natural origin, such as blood, or material produced by recombinant cell cultures, with a risk of introduction of pathogens like viruses or prions into a product to be administered to human patients.

Moreover, because of the robust nature of the PVA particles provided as hemostats in the present hemostatic product, the product/particles can be subjected to sterilization methods, such as γ-radiation sterilization treatment, without the risk of losing their hemostatic properties or physicochemical features helpful in acting as a hemostat, such as absorption and swelling characteristics.

The present invention is drawn to a final hemostatic product suitable for the administration to a human patient comprising a biocompatible hemostatic material comprising or consisting of non-porous, cross-linked polyvinyl alcohol particles (PVA particles) which can be commercialized in an appropriate dosage design suitable for storage and marketing on the pharmaceutical market. Due to the stability and the synthetic nature of the product according to the present invention it is extremely storage-stable and does not need cooling or freezing.

The present product is provided as a product comprising the PVA particles. As also disclosed in the example section, the selection of the diameter of the particles can result in an advantageous embodiment of the present invention. In the course of the present invention, it could be shown that a mixture of various particle sizes is beneficial for the hemostatic performance of the products according to the present invention. Whereas products wherein only very small particles are present have a tendency to stronger adhere to gauze than to the wound and products wherein only large particles are present are less successful in hemostasis by showing oozing bleedings, compositions wherein large and small particles are mixed together show excellent hemostatic properties, even in very challenging bleeding models, such as the porcine liver punch model used in the example section. This mixture provides a balanced approach for achieving a good surface-to-volume ratio of the product as a whole and preventing an unintended adhesion to other material (such as gauze) or preventing formation of a gelled barrier layer. Accordingly, a preferred hemostatic product according to the present invention contains or consists of particles with a diameter of less than 500 μm (or at least as a preparation with the majority of the particles having a diameter of less than 500 μm) and is preferred due to the better performance in hemostasis. Accordingly, a hemostatic product according to the present invention, wherein at least 50% of the PVA particles have a diameter of less than 500 μm, preferably wherein at least 80% of the PVA particles have a diameter of less than 500 μm, especially wherein 100% of the particles have a diameter of less than 500 μm, represents a preferred embodiment of the present invention.

In view of this preferred mixing of small and large particles, in a preferred embodiment, the hemostatic product according to the present invention contains PVA particles wherein the ratio of PVA particles with a diameter of less than 200 μm to PVA particles with a diameter between 200 and 500 μm is from 10:90% (w/w) to 90:10% (w/w), preferably from 15:85% (w/w) to 85:15% (w/w), especially from 20:80% (w/w) to 80:20% (w/w).

The products according to the present invention are biocompatible and biodegradable. The degradation capabilities of the product can e.g. be adjusted by selecting the functionality of the PVA molecules and the crosslinkers. The PVA particles of the hemostatic product according to the present invention are preferably adjusted so that they are absorbable to more than 90% by a human patient's body within 6 months after administration, preferably within 3 months, more preferred within 2 months, especially within 1 month, after administration to said human patient.

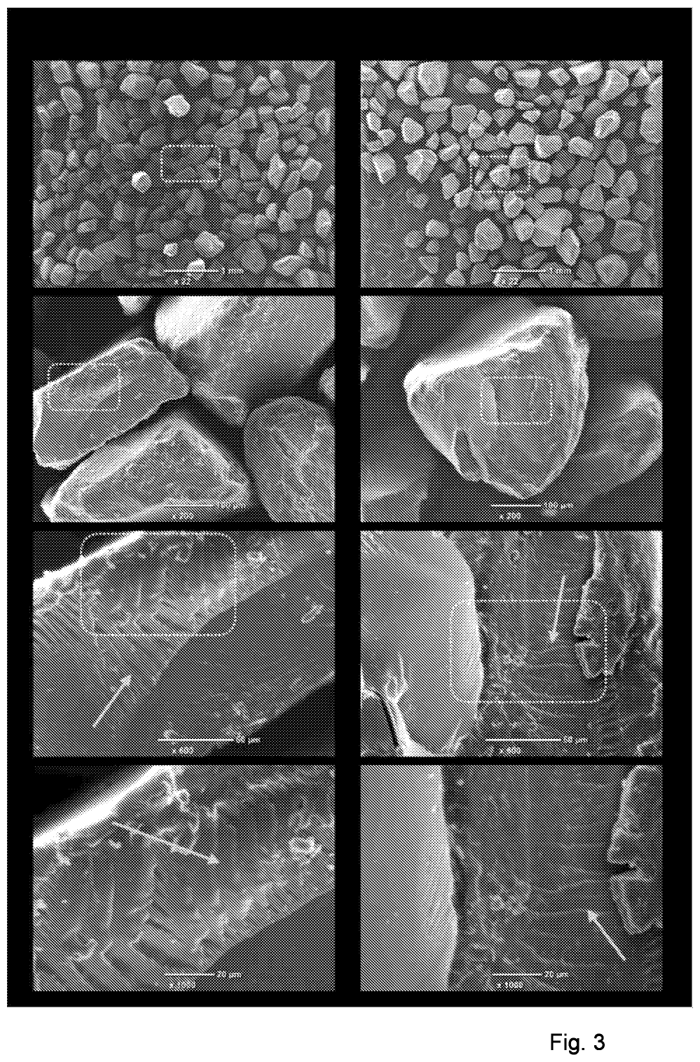

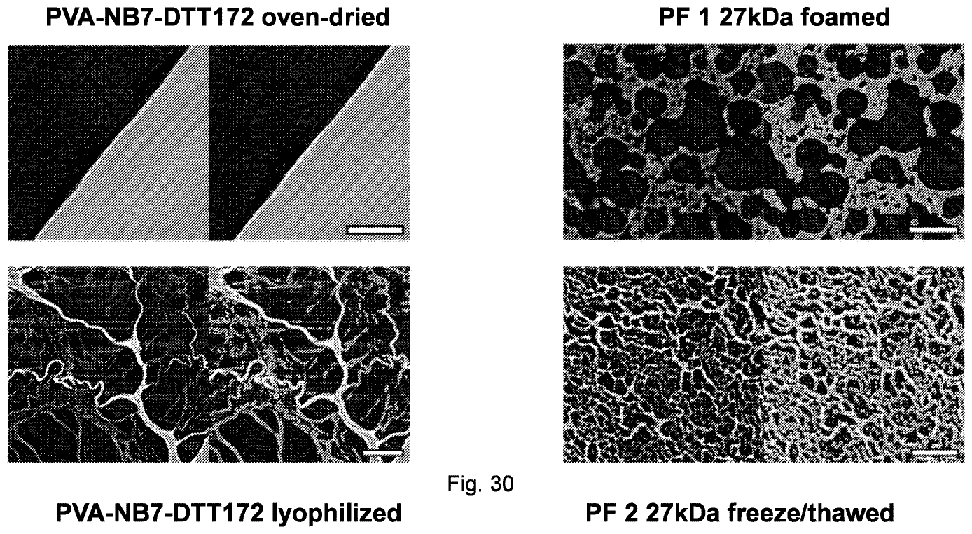

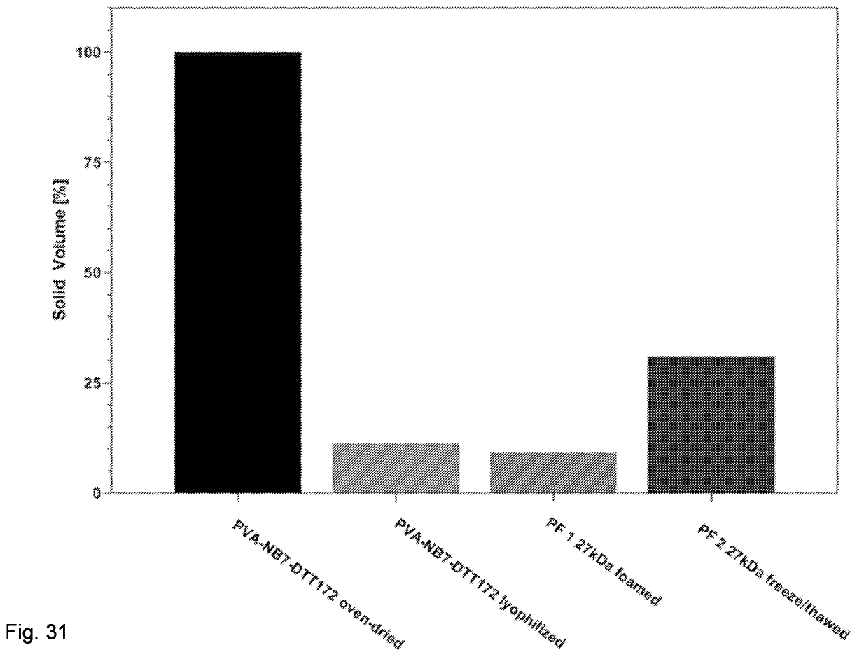

As already mentioned above, the PVA particles according to the present invention are of a non-porous nature (see also FIG. 3). This lack of porosity may be evidenced by determining a porosity level of the PVA particles according to the present invention, wherein the lack of porosity is defined as having a solid volume of at least 70%, preferably at least 80%, more preferred at least 90%, especially at least 95%, as determined by a nano-CT measurement, preferably as determined by intensity-based density calculation (in %). The preferred method for defining the exact porosity (in % of total volume) by intensity-based density calculation is disclosed in the example section (see also FIGS. 30 and 31), wherein the method was created for the present invention, based on the correlation of porosity and attenuation in CT. Similar methods based on the same principles were also reported by Withjack et al. (SPE Formation Evaluation, 3 (1988), 696-704; doi: 10.2118/16951-PA), Taud et al. (J. Petrol. Sci. Engin. 47 (2005), 209-217) and Bruns et al., Adv. Water Resour. 107 (2017), 32-42). Measurements of intensity-based density calculation are preferably carried out on Scanco uCT 50, wherein the resulting scans are analyzed based on a method based on the correlation between porosity and attenuation and intensity in CT, to give the proportion of non-filled volume to filled solid volume. The PVA particles as shown in FIGS. 30 and 31 showed to be of 100% solid volume.

Due to the fact that the PVA particle according to the present invention have already an activity as a passive hemostat, these particles do not need to be combined with other hemostatic agents, such as coagulation factors or other coagulation active agents (such as in the compound according to WO 2017/037178 A1). This enables a very safe product which is not exposed to the risk compounds from natural sources or from recombinant cell cultures (such as pathogens, like viruses or prions). Accordingly, the hemostatic product according to the present invention can preferably be kept “clean” from such compounds by providing the PVA particles free of covalently or non-covalently bound blood compounds of natural or recombinant origin, or functional fragments of such blood compounds. Preferably, the hemostatic product according to the present invention is therefore free of bound blood compounds of natural or recombinant origin, or functional (=hemostatic) fragments of such blood compounds, such as thrombin, fibrinogen, TRAP peptides or functional fragments thereof.

The PVA particles according to the present invention may be substituted with a variety of chemical compounds (see below and in the example section). Preferably, the PVA particles according to the present invention have a certain substitution degree. Preferred substitution degrees differ from a total (100%) substitution and a substitution below 1% so that the PVA particles according to the present invention may have a substitution degree of 2% to 70%, preferably of 3% to 55%, more preferred of 4% to 50%, especially of 5% to 40%.

Preferably, the PVA particles have a substitution degree of 1% or more, preferably of 2% or more, especially of 3% or more.

According to a preferred embodiment of the present invention, the PVA particles are non-porous, cross-linked norbornene-group-modified particles (PVA-NB particles), preferably ionic PVA-NB particles, especially PVA-NB particles made with carbic anhydride.

As used herein, especially in the example section, PVA particles which are obtained after hydrogel crosslinking, drying and milling were named after the following Scheme: PVA-XXaa-YYbb.
XX . . . pendant group on PVA (added by modification of PVA, e.g. “NB” for norbornene from carbic anhydride)
YY . . . multifunctional thiol for photocrosslinking (e.g. DTT, GDMA, or ETTMP)
aa . . . degree of modification in %, relative to repeating units of PVA
bb . . . molar thiol/ene ratio for crosslinking in %

The PVA particles are preferably PVA-NB particles wherein crosslinking was performed by thiol groups, preferably by dithiothreitol (DTT), glycoldimercapto acetate (GDMA), glycoldimercapto propionate (GDMP), dithiol-functionalized poly(ethylene glycol) (HS-PEG-SH), Tris [2-(3-mercaptopropionyloxy)ethyl]isocyanurate (TEMPIC), pentaerythritol tetramercaptoacetate (PETMA), pentaerythritol tetramercaptoproprionate (PETMP) or ethoxylated trimethylolpropane tri (3-mercaptopropionate) (ETTMP), especially by DTT or GDMA. These crosslinkers have shown to result in specifically advantageous PVA-NB particles according to the present invention. The degree of crosslinking of the reactive groups is variable. From this, it follows that the crosslinking degree, e.g. 100% crosslinking or 50% crosslinking, is usually not critical.

Hydrogels can swell by taking up water molecules until elastic forces between the polymer chains, e.g. covalent crosslinks, restrict further uptake and an equilibrium is reached. Therefore, the degree of crosslinking directly influences the swelling behavior of materials (Peppas et al., (Ann. Rev. Biomed. Engin. 2 (2000), 9-29), Flory et al. (J. Chem. Phys. 11 (1943), 512-520), Flory et al. (J. Chem. Phys. 11 (1943), 521-526)).

The degree of crosslinking for the PVA-NB system can be tailored for example by the degree of substitution of PVA polymer chains with norbornene pendant groups and (subsequently) also by the amount of multifunctional thiol-crosslinker in relation to available norbornene groups in the formulation. Establishing such hydrogels is well available for the person skilled in the art; suitable methods are disclosed e.g. in Baudis et al. (J. Polymer Sci. Part A: Polymer Chem. 54 (2016), 2060-2070).

In order to provide the balanced approach mentioned above with respect to mixing small and large PVA particles, a preferred hemostatic product according to the present invention contains or consists of PVA particles having an average particle size of 1 to 1000 μm, preferably of 5 to 500 μm, especially of 10 to 300 μm. The average particle size may be determined according to the international standard on presenting various characteristic particle sizes, the ISO 9276 (representation of results of particle size analysis). This set of various average sizes includes median size, geometric mean size, average size. In the selection of specific small-size particles is common the use of ISO 565 and ISO 3310-1 to the choice of mesh size. The product according to the present invention may contain a variety of particle sizes comprising a portion of large particles (200 to 1000 μm, preferably 200 to 500 μm, in size) and a portion of smaller particles (below 500 μm, preferably below 200 μm). According to a preferred embodiment, the product according to the present invention contains at least 20%, preferably at least 30%, especially at least 40%, of PVA particles with a size of 100 to 200 μm and at least 20%, preferably at least 30%, especially at least 40%, of PVA particles with a size of 200 to 500 μm.

According to a specifically preferred embodiment, the product according to the present invention contains 20 to 70%, preferably 30 to 60%, of PVA particles with a size of 100 to 200 μm and of 10 to 60%, preferably 20 to 50%, of PVA particles with a size of 200 to 500 μm. If not explicitly stated otherwise, “size” refers to the size effective in sieving cut-offs (in typical steel mesh sieves, as also used in the examples), e.g. particles having a size of 500 μm or more are not passing a sieve with a cut-off of 500 μm; particles with a size of below 200 μm pass a sieve with a cut-off of 200 μm.

Preferred hemostatic products according to the present invention are further defined by the swelling capacity of the PVA particles. As demonstrated in the example section, the swelling capacity (as well as the absorption capabilities) can be adjusted e.g. by appropriate choice of PVA modifications and/or crosslinkers. Accordingly, preferred PVA particles in the present hemostatic product have a swelling capacity of at least 100% relative volume increase after 60 minutes, preferably of 150% to 1000% relative volume increase after 60 minutes, especially of 200% to 500% relative volume increase after 60 minutes; and/or a mass swelling capacity of 100% to 1000% relative mass increase after 24 h, preferably a mass swelling capacity of 200% to 800% relative mass increase after 24 h, especially a mass swelling capacity of 250% to 600% relative mass increase after 24 h.

Since the products according to the present invention can be set up as a fully synthetic product based on PVA, the product is very stable. Quite in contrast to reports in the literature, the products according to the present invention also show a high stability and a significant resistance with respect to sterilization methods, especially irradiation, such as γ-radiation. It was shown that such radiation did not have a significant (negative) effect on the major properties of the products according to the present invention, such as hemostatic activity, swelling properties or cytotoxicity. Accordingly, the hemostatic product according to the present invention and/or the PVA particles contained therein is/are aseptic by γ-radiation sterilization treatment.

The hemostatic product according to the present invention is provided as a final product ready for purchase e.g. in pharmacies or wholesale trade. Accordingly, the products according to the present invention may be provided as products having been produced by GMP (good manufacturing practice) standards required by EMA or US FDA and as products which are appropriately wrapped; i.e, wherein the hemostatic product is sterile, preferably wherein the product is wrapped in a sterile container or bag.

According to a preferred embodiment, the hemostatic product according to the present invention does not comprise an additional coagulatively active agent further to the PVA particles, i.e. the PVA particles are the only hemostatic agent contained in the product.

Preferably, the hemostatic product according to the present invention may be provided as a single product or as part of a composite hemostatic product or hemostatic kit, preferably wherein the composite product is a conglomerate, a particulate, a granulate material; and/or wherein the PVA particles are coated, mixed or impregnated with an active agent, preferably an anti-bacterial agent, a coagulatively active agent, an immunosuppressive agent, an anti-inflammatory agent, an analgesic agent, an anti-fibrinolytic agent, a growth factor, a vitamin, or mixtures thereof.

According to a preferred embodiment, the PVA of the hemostatic product according to the present invention has a molecular weight of 3 to 100 kDa, preferably of 5 to 70 kDa, especially of 9 to 40 kDa.

According to a specific aspect, the present invention also relates to the present hemostatic product, for use in human or animal therapy and/or surgery, preferably for use in hemostasis as a passive hemostat, especially for use in wound treatment.

The present invention also relates to a method for the treatment of a wound or for achieving hemostasis in a human patient, wherein an effective amount of a hemostatic product according to the present invention is administered to a patient with a wound or in need of hemostasis. Preferably, the product is administered in dry form (eventually mixed/coated/impregnated (etc.) with other components in dry from, such as powdered further substances). In certain embodiments, the hemostatic product may even be mixed with a pharmaceutically acceptable aqueous solution before administration.

The present invention also discloses methods for producing the PVA particles according to the present invention.

The present invention also provides a method for producing a hemostatic product comprising cross-linked non-porous PVA particles, preferably for producing the hemostatic material according to the present invention, wherein polyvinyl alcohol is covalently modified with a modifying agent comprising at least one of an modifying agent comprising an “ene” and/or “yne” group, preferably norbornene, maleimide, 1,2-substituted alkenes and alkynes, allyl and vinyl group (e.g. respective esters, ethers and carbonates), acrylate, methacrylate, propynyl; cross-linked to form PVA particles, and then optionally washed and dried; without applying a step causing a porous structure to be formed; and preferably without applying a lyophilization step.

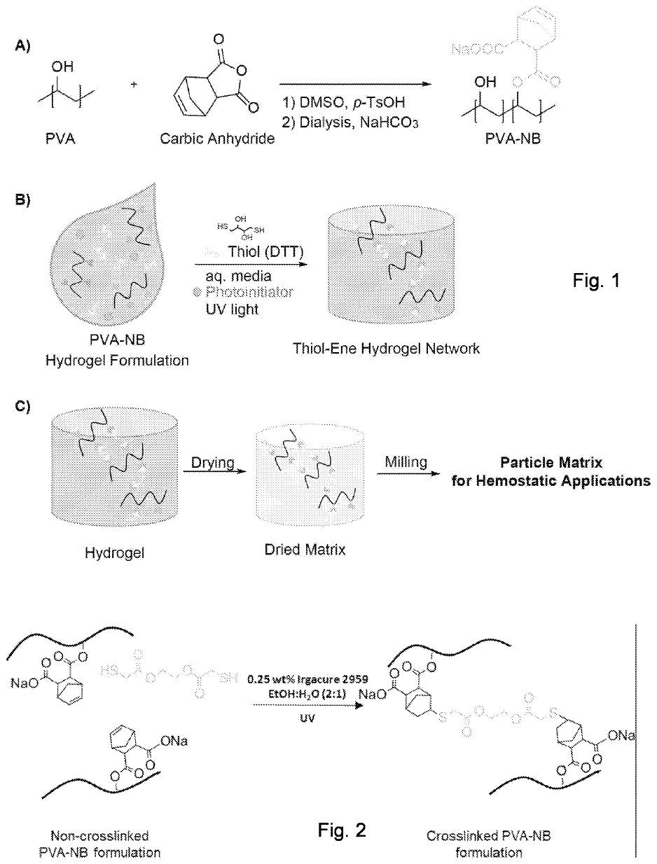

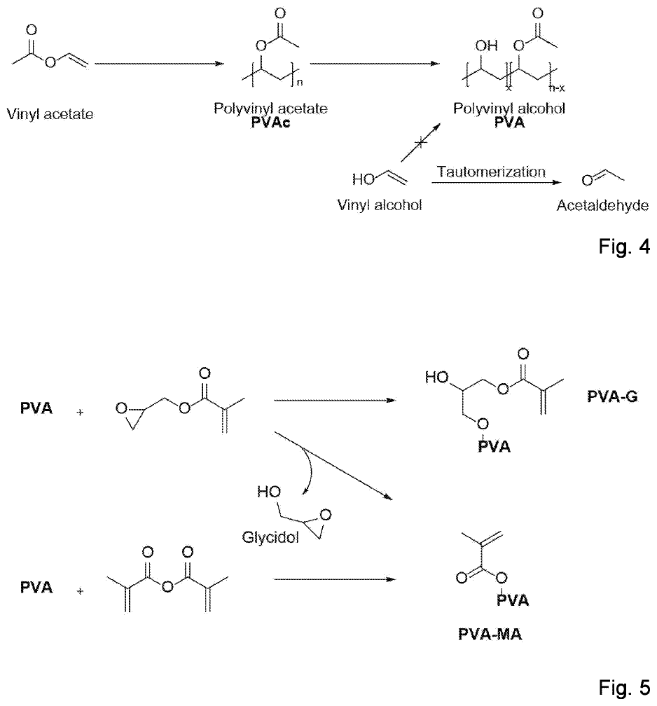

A preferred method for producing the PVA particles according to the present invention relies on crosslinking by UV-initiated thiol-ene chemistry (see FIG. 1). Crosslinking according to the present invention is therefore performed preferably in suitable UV irradiation settings (e.g. INTELLI RAY 600 from Uvitron International, or Omnicure S2000 lamp). Crosslinking using other suitable strategies is also possible and may include alternative initiator systems (e.g. azo compounds, persulfates or other peroxides, metal ions). Polyvinyl alcohol cannot be directly synthesized from its corresponding vinyl alcohol monomer. Vinyl alcohol undergoes tautomerization to the more stable acetaldehyde, preventing “direct” PVA synthesis from vinyl alcohol. Free-radical polymerization of vinyl acetate yields polyvinyl acetate that can then be hydrolyzed to polyvinyl alcohol, a hydrophilic, FDA-approved vinyl polymer. These processes are outlined in FIG. 4.

Many PVA precursors are available commercially with varying molecular weight and degree of hydrolysis (i.e. residual acetyl groups along the backbone). A preferred PVA variant used according to the present invention only has a small number of residual acetyl groups of 5% or less, preferably of 4% or less, especially of 3% or less (the material used in some of the examples has a number of residual acetyl groups of ~2%).

According to the degree of polymerization and degree of hydrolysis (degree of saponification), different varieties of PVA are commercially available. Degree of saponification and hydroxyl content directly affect mechanical and physico-chemical properties of PVA materials.

The high number of available hydroxyl groups in PVA makes it an attractive and versatile polymer platform for functionalization and crosslinking. PVA finds applications in many different fields from packaging and coatings to contact lenses, adhesives or pharmaceuticals.

Degradation of PVA-based materials can take place via different mechanisms depending on many factors like chemical crosslinking, degree of saponification, molecular weight or crystallinity. PVA is degraded at high temperatures via an elimination mechanism: H₂O is eliminated, leading to C—C double bond formation in a conjugated pattern along the backbone. Further degradation can take place via chain scissions. Additional to thermal degradation, enzymatic processes in microorganisms are known to degrade PVA. In biomedical application settings relevant conditions for these degradation mechanisms are not met. Therefore, both thermal and microbial degradation of PVA backbones do not play significant roles. Degradation in physiological environments mainly takes place via the elimination of crosslinks in the network and eventual dissolution of PVA chains. Studies showed clearance of PVA from the body through the kidneys (urine) and the liver (feces). Therefore, the type and number of crosslinks introduced to the material must be considered as a key factor when it comes to material degradation.

Due to the structure of PVA, multiple strategies exist to produce PVA-derived hydrogels via crosslinking. A list of crosslinking methods is provided with examples from literature: The high number of hydroxyl functionalities and structure allows the formation of hydrogels by means of non-covalent interactions between the chains. These “physical crosslinks” can for example be introduced by repeated freeze-thaw-cycling of PVA-containing solutions. Crystalline sections are introduced due to strong physical interactions between the PVA-polymer chains (Peppas et al., J. Appl. Pol. Sci. 20 (1976), 1457-1465; Stauffer et al., Polymer 33 (1992), 3932-3936; Tamer et al., Sci. Rep. 11 (2021), 3428). PVA can be crosslinked and/or precipitated by addition of salts, e.g. sodium sulfate or borates (Patachia et al., Express Polymer Letters 2009, 3, 320-331; Curley et al., J. Mech. Behav. Biomed. Mat. 40 (2014), 13-22; Cencetti et al., Carboh. Polym. 90 (2012), 1362-1370; Chen et al., J. Appl. Polym. Sci. 132 (2015): 22; Miyazaki et al., Polymer 51 (2010), 5539-5549). Gamma irradiation can be used for crosslinking of PVA chains (Lugao et al., Nucl. Instr. Meth. Phys. Res. Section B: Beam Interact. Mat. Atoms 185 (2001), 37-42; Nikolic et al., Electrochem. Comm. 9 (2007), 2661-2665; El Salmawi et al, J. Macromol. Sci. Part A 44 (2007), 541-545). PVA can be blended with a wide range of other synthetic or natural polymers or form composites with inorganic materials (Neres Santos et al., Materials (Basel) 2019, 12 (4), 559; Kamoun et al., Arab. J. Chem. 2015, 8 (1), 1-14; Kamoun et al., J. Adv. Res. 2017, 8 (3), 217-233; Liu et al., J. Biomed. Mat. Res. Part B: Appl. Biomat. 90B (2009), 492-502.): alginate (Bahadoran et al., Sci. Rep. 10 (2020), 7342; Tarun et al., in: Calcium alginate/PVA blended nano fibre matrix for wound dressing, 2012), chitosan (Baghaie et al., J. Biomat. Sci., Polymer Ed. 28 (2017), 2220-2241; Massarelli et al., Int. J. Pharm. 593 (2021), 120110; Don et al., Carboh. Polym. 63 (2006), 331-339; Fan et al., Carboh. Polym. 146 (2016), 427-434), various other carbohydrate polymers (Baghaie et al., J. Biomat. Sci., Polymer Ed. 28 (2017), 2220-2241; Wang et al., Mat. Sci. Engin. C 69 (2016), 268-275; Kenawy et al., Arab. J. Chem. 7 (2014), 372-380), gelatin (Ino et al., J. Biomed. Mat. Res. Part B: Appl. Biomat. 101 (2013), 1549-1559; Pawde et al., J. Appl. Polym. Sci. 109 (2008), 3431-3437; Yang et al., J. Mat. Chem. B 9 (2021), 1568-1582), PVP (Nho et al., J. Appli. Polym. Sci. 85 (2002), 1787-1794; Thongsuksaengcharoen et al., ACS Omega 5 (2020), 25973-25983), PNIPAAm (Cheng et al., Sensors and Actuators B: Chemical 255 (2018), 3117-3126; Fathi Azarbayjani et al., A.; Pharm. Sci. 13 (2010), 400-410; Zhang et al., Acta Biomat. 5 (2009), 488-497), montmorillonite clay (Kokabi et al., Eur. Polym. J. 43 (2007), 773-781) or ZnO nanoparticles (Baghaie et al., J. Biomat. Sci., Polymer Ed. 28 (2017), 2220-2241).

Other chemical strategies for crosslinking of PVA are mostly based on multifunctional hydroxyl-reactive molecules that act as crosslinking agents or are based on the synthesis of chemically modified PVA-variants via a modifying agent. Chemical modification strategies for PVA-crosslinking introduce reactive groups that can later be used for crosslinking reactions. For example, C—C double bonds can be introduced for subsequent free-radical polymerization or Click chemistry approaches like thiol-ene-based crosslinking or Diels-Alder mechanism but also other mechanisms have been exploited. Crosslinking agents that are reactive towards OH-groups of unmodified PVA can for example include:
aldehydes (e.g. glyoxal, glutaraldehyde, oxidized carbohydrate like cellulose to yield respective dialdehydes);
carboxylic acids, carboxylic anhydrides, acyl halides;
epoxides and alkyl halides for etherifications (e.g. epichlorohydrin and PEG diglycidyl ether);
isocyanates (e.g. hexamethylene diisocyanate, isocyanate prepolymers or blocked isocyanates);
sulfones;
orthosilicates (e.g. tetraethyl orthosilicate);
free-radical polymerization with vinyl monomers like N,N′-methylenebisacrylamide or crosslinking with vinyl silanes;
modification with DOPA for pH- and oxidation-induced crosslinking;
modification of PVA with methacrylate groups via glycidyl methacrylate as shown in FIG. 5. Depending on reaction conditions this either leads to a ring-opening reaction of the epoxide functionality or a transesterification reaction releasing glycidol. Methacrylated PVA can be crosslinked via free-radical-chain reactions using initiators. Substances that generate radicals for example under thermal stress or during UV exposure can be used for curing. Alternatively, methacrylation of PVA can also be achieved by reaction with methacrylic anhydride as shown in FIG. 5. Methacrylated PVA was also used for Michael-addition crosslinking;
modification with acryloyl chloride in DMF to obtain PVA with pendant acrylate functionalities for later radical-induced crosslinking.
modification with norbornene groups for later thiol-ene follow-up chemistry for crosslinking or biofunctionalization, also in combination with thiolated PVA;
modification with allyl groups for thiol-ene crosslinking
modification for Copper (I)-catalyzed azide-alkyne cycloaddition (“CuAAC”)
modification of PVA with aldehyde and hydrazide functionalities for hydrazone-bond-mediated crosslinking.

The dry particle matrix according to the present invention has proven the great hemostatic potential of this product variant produced from norbornene-group-modified PVA (“PVA-NB”) that was crosslinked via a thiol-ene reaction using dithiothreitol (DTT) as crosslinker. This was attributed to two factors: Firstly, the used PVA-NB-DTT material's highly hydrophilic network with high soaking capabilities. This polymer network is able to absorb high amounts of an aqueous phase in a short period of time and is therefore well suited for application as passive hemostatic matrix. This means that by virtue of its absorption properties the material according to the present invention is able to exert a concentrating effect on blood at the wound site leading to a locally increased concentration of blood cells, proteins and hemostatic factors.

Secondly, the PVA particles according to the present invention, especially the particles with the PVA-NB backbone of the network, preferably carry multiple anionic charges on the one hand attributing the hydrophilic properties to the material and on the other hand conferring contact-activation properties to the material.

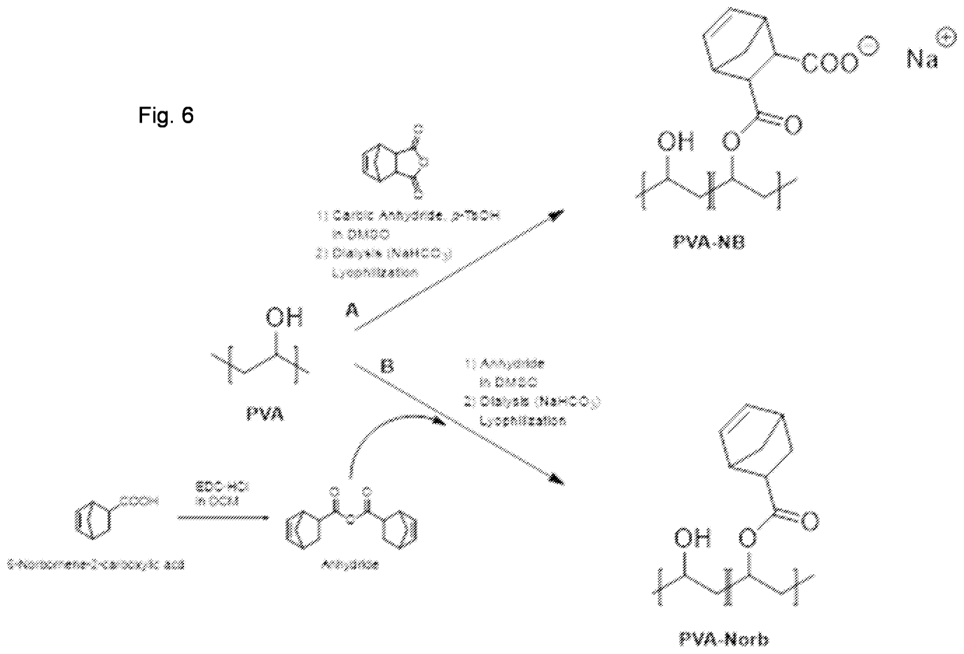

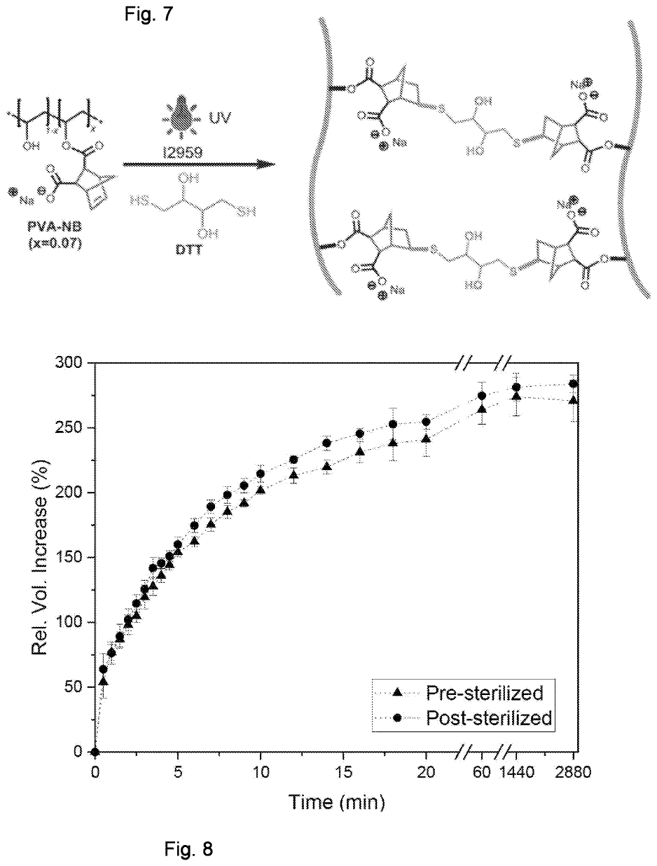

An ample set of PVA-norbornene modifications can be synthesized as examples of this family of poly(vinyl alcohol)-based networks exploiting thiol-norbornene step-growth mechanism. For example, the degree of norbornene-group substitution along the linear polymer backbone before crosslinking can be varied. Furthermore, norbornene modification of the polymer is possible through different strategies. For example, the reaction with carbic anhydride provides a facile method that results in a linear, hydrophilic, poly-anionic polymer modification after neutralization due to the presence of added carboxylate functionalities. Non-ionic PVA-norbornene variants can also be obtained for example by esterification strategies using 5-norbornene-2-carboxylic acid (resulting in “PVA-Norb”). An overview of both example synthetic strategies can be seen in FIG. 6.

Modification of PVA with pendant norbornene groups for subsequent thiol-ene chemistry yields a polymer termed “PVA-NB” (see FIG. 6, reaction path A). PVA-NB was yielded preferably in a reaction with carbic anhydride in dry DMSO with catalytic amounts of para-toluenesulfonic acid (p-TsOH); Baudis et al., J. Polym. Sci. Part A: Polym. Chem. 54 (2016), 2060-2070). The achieved degree of substitution of PVA could be regulated by according changes to the used amount of carbic anhydride (i.e. the norbornene group precursor used for the modification reaction) for the reaction.

Additionally, PVA-Norb was synthesized as non-ionic variant. The obtained macromer was termed “PVA-Norb” (see FIG. 6, reaction path B). The strategy was to create a reactive intermediate of 5-norbornene-2-carboxylic acid (i.e. carboxylic anhydride) that can be isolated for further transformations. Firstly, a carboxylic anhydride was generated from 5-norbornene-2-carboxylic acid (i.e. 5-norbornene-2-carboxylic anhydride) using EDC-coupling chemistry in DCM as solvent.

The isolated anhydride may be used for modification of PVA (e.g. in DMSO to yield PVA-Norb after dialysis and lyophilization with a degree of substitution of 5% of repeating units of PVA being modified with a pendant non-ionic norbornene group).

Therefore, the present invention also relates to a method for producing a hemostatic product comprising cross-linked non-porous PVA particles, preferably for producing the hemostatic material according to the present invention, as disclosed above, wherein modification of PVA according to the present invention can be achieved by reaction with a modifying reagent comprising of at least a chemical moiety suitable for covalent coupling to polyvinyl alcohol or a crosslinking agent. More specifically, the modifying agent may also include one or more reactive groups that allow subsequent reactions like crosslinking via the introduced chemical moiety of the modifying agent itself, crosslinking through subsequent addition of crosslinking agents, subsequent crosslinking with crosslinking agents through reactions with the modifying agent groups or subsequent functionalization. For example, the modifying agent may be comprising one or multiple of the following functionalities for covalent bond formation with the PVA chains: oxyacids and esters and anhydrides thereof, carboxylic acids, carboxylic anhydrides, acyl halides, alkyl halides, epoxides, isocyanates, isothiocyanates, aldehydes for covalent modification or directly crosslinking the polymer chains. In addition, the modifying agent may be comprising one or multiple “ene” and/or “yne” groups, preferably norbornene, maleimide, 1,2-substituted alkenes and alkynes, allyl or vinyl group, acrylate, methacrylate, propynyl for subsequent functionalization or crosslinking. According to a preferred embodiment, the modifying agent is carbic anhydride. Carbic anhydride as a “modifying agent” according to the present invention has one of both groups: A) A chemical moiety that has reactivity towards the PVA chains (i.e. internal anhydride of a dicarboxylic acid) that could theoretically, under certain circumstances, also be used to directly crosslink the polymer chains; and B) the norbornene group as ene functionality for subsequent reactivity for crosslinking (in this case with multifunctional thiols) or other functionalizations.

Cross-linked PVA may be used to form PVA particles including optional washing and drying procedures; without applying a step causing a porous structure to be formed, preferably without applying a lyophilization step.

Preferably, the polyvinyl alcohol used as a starting material has a number of residual acetyl groups of 5% or less, preferably of 4% or less, especially of 3% or less.

According to a preferred embodiment, the PVA used in the methods according to the present invention has a molecular weight of 3 to 100 kDa, preferably of 5 to 70 kDa, especially of 9 to 40 kDa.

According to a preferred embodiment, in the present method the cross-linked polyvinyl alcohol is milled to obtain cross-linked polyvinyl alcohol particles, preferably particles with an average particle size of 1 to 1000 μm, preferably of 5 to 500 μm, especially of 10 to 300 μm.

As outlined above, it is preferred if the polyvinyl alcohol is chemically modified with a modifying agent as described above, preferably including oxyacids; mono- or multifunctional carboxylic acids; or esters or anhydrides, acyl halides, N-hydroxysuccinimidyl esters, or O-acylisoureas thereof; alkyl halides, epoxides, isocyanates, isothiocynates aldehydes; especially carbic anhydride, oxalic acid, succinic acid, or succinic anhydride OBJ

Preferably, the product and/or the PVA particles is/are sterilized, preferably sterilized with γ-irradiation, especially with γ-irradiation of a dose of 10 to 40 kGy. As also shown in the example section of the present invention, even such γ-irradiation doses have no significant impact on the relevant features of the product according to the present invention.

In a specifically preferred method, polyvinyl alcohol is modified with norbornene groups, preferably by reacting polyvinyl alcohol with carbic anhydride, especially in dry DMSO with catalytic amounts of para-toluenesulfonic acid; to obtain a modified polyvinyl alcohol, and wherein the modified polyvinyl alcohol is cross-linked by UV-initiated thiol-ene chemistry, preferably by a radical-mediated thiol-ene step growth crosslinking procedure, especially in the presence of dithiothreitol and a photoinitiator.

According to a preferred embodiment, the PVA particles are milled and preferably sieved through a particle sieve with a cut-off of 100 μm to 1 mm, especially with a cut-off of 200 to 500 μm.

According to another aspect, the present invention also relates to a non-porous, biocompatible hemostatic material comprising cross-linked polyvinyl alcohol, obtainable by a method according to the present invention.

According to another aspect, the present invention also relates to a kit, preferably for use in surgery or in the treatment of injuries or wounds, comprising a hemostatic product according to the present invention, and further comprising at least one administration device, buffer solution, syringe, tube, catheter, forceps, scissors, sterilizing pad or lotion.

Preferably, the buffer solution further comprises a component selected from the group consisting of an anti-bacterial agent, a coagulatively active agent, an immunosuppressive agent, an anti-inflammatory agent, an analgesic agent, an anti-fibrinolytic agent, a growth factor, a vitamin, a cell, or mixtures thereof.

Preferably, however, the products according to the present invention are provided in dry form and are only subjected to contact with aqueous environments (wound fluids, buffers, etc.) at the time of administration to the patient (i.e. at the surgical desk, in an emergency situation (as first aid and/or in emergency situations)). Therefore, a preferred embodiment of the kit or the product according to the present invention is a combination of the PVA particles according to the present invention with powders of another active agent, preferably an anti-bacterial agent, a coagulatively active agent, an immunosuppressive agent, an anti-inflammatory agent, an analgesic agent, an anti-fibrinolytic agent, a growth factor, a vitamin, or mixtures thereof.

Methods of use of disclosed embodiments can comprise application to a site where bleeding is desired to be reduced. For example, disclosed methods comprise application of disclosed materials, slurries, granules, powders, or substrates to a site where bleeding is desired to be reduced, such as a site of injury or surgical procedure. Thus, disclosed embodiments provide an excellent tool for sealing of anastomosis, for suture line sealing, and to safeguard hemostasis in resection sites. These methods are further described in the following Examples.

The following non-limiting Examples are provided for illustrative purposes only to facilitate a more complete understanding of representative embodiments. These examples should not be construed to limit any of the embodiments described in the present specification.

### EXAMPLES

Polyvinyl alcohol (PVA) materials according to the present invention were crosslinked by means of UV-initiated thiol-ene chemistry. To this end, reactive hydroxyl functionalities of PVA were chemically modified by reaction with carbic anhydride. This reaction yields PVA-NB as polymer modification, which is a linear, water-soluble PVA backbone, modified with norbornene groups. The C—C double bond of norbornene (also termed “ene”) is highly reactive towards radical-mediated thiol-ene step-growth crosslinking. Therefore, this modification facilitates efficient UV-crosslinking of the polymer to obtain hydrogel-based biomaterials. After crosslinking, the materials were washed and dried to obtain the dry matrix material which was then milled to obtain particle matrices for applications in local hemostatic applications. This procedure is illustrated schematically in FIG. 1.

PVA particle prototypes were developed and successfully tested in a preclinical model. Found results showed that PVA-based biomaterials according to the present invention are a suitable material class for hemostatic applications.

The present examples also show preclinical tests of the PVA particle based synthetic hemostats according to the present invention. Moreover, the impact of sterilization processes (e.g. γ-radiation sterilization) on material properties of PVA-based particles was investigated. Pre- and post-sterilized samples produced and sterilized via γ-radiation sterilization were tested for their volumetric and mass swelling, respectively, as well as for their in vitro degradability and cytotoxicity. It was found that the sterilization process employing γ-radiation did only have minor effects on investigated material properties. Mass and volumetric swelling results only showed small differences, especially regarding short-term volumetric swelling. No cytotoxicity was found for both, pre- and post-sterilized materials using a direct BrdU cytotoxicity assay. A degradability study of pre/post sterilized material on particle in vitro degradation showed full degradation of the particles (sterilized and non-sterilized) after 5 weeks.

The influence of crosslinking of PVA particles according to the present invention was investigated at three points of variation: Type of chemical modification of PVA, type of thiol crosslinker, amount of thiol crosslinker.

Additional to differences in network chemistry, the influence of particle size variation was investigated. Unmodified and non-crosslinked PVA was included in the present examples as a reference material.

Model particle materials were produced and characterized regarding their swelling properties as important characteristic of a passive particle hemostat.

Selected prototype materials were additionally tested in an in vivo bleeding model.

### Example 1

PVA Modification with Norbornene Groups

PVA (27 kDa, degree of hydrolysis 98%, 10.00 g) and para-toluenesulfonic acid (0.02 g) were dissolved in 60 mL of DMSO at 70 °C (Ar atmosphere). 5-norbornene-2,3-dicarboxylic anhydride (carbic anhydride, 2.80 g) was dissolved in 30 ml DMSO and added dropwise to the stirred PVA solution at 60 °C and the resulting solution was stirred for about 16 h. The solution was dialyzed against aqueous sodium bicarbonate and deionized water. The polymer modification, termed PVA-NB, was obtained via lyophilization of the dialyzed solution as a white solid with excellent water solubility.

The product was characterized via proton NMR spectroscopy regarding its degree of substitution (DS) as percentage of norbornene moieties per CH group of PVA. Material with DS of about 7% was obtained by this method.

An alternative work-up of the polymer modification reaction includes precipitation of the modified polymer: After the esterification reaction in DMSO, the DMSO solution was precipitated by slow addition to a large excess of distilled acetone. The precipitated polymer was re-dissolved in deionized water, the pH was adjusted with sodium bicarbonate to ~7, and this aqueous solution was again precipitated by slow addition to a large excess of distilled acetone. The precipitated polymer was re-dissolved in deionized water and precipitated in acetone two more times until a DMSO-free product was obtained by drying procedures (vacuum oven, 50 °C, 50 mbar).

### Example 2

### Hydrogel Synthesis

50% of available norbornene groups of PVA-NB were used for crosslinking with DL-dithiothreitol (DTT). More specifically, a stock solution of 0.5 wt % Irgacure® 2959 (α-hydroxy-4-(2-hydroxyethoxy)-α-methylpropiophenon) in PBS (pH=7.4) was prepared. This solution was used to prepare a solution of PVA-NB (DS~7%) and appropriate amounts of DTT. DTT solution was mixed with the macromer solution to result in a total macromer concentration of 20 wt % and a molar ratio of thiol/norbornene of 50%. This formulation was then photocrosslinked to yield hydrogel pellets.

### Example 3

### Milling of Hydrogel Matrices

Cured hydrogels were washed with deionized water for 24 h and water was changed three times. Afterwards, water was decanted, and the swollen hydrogels were subjected to a de-swelling step by placing them into distilled ethanol for 12 h (2 ethanol changes). Hydrogels were pre-dried at 50 °C for 24 h and dried at 60 °C and 50 mbar to constant weight before milling. Dried hydrogels were milled with a Retsch ZM200 centrifugal mill to yield particles. The mill was operated at 10000 rpm, using the 12-prism-blade rotor and trapezoid-hole sieves with spacers towards the blade rotor. The material was subjected to multiple milling steps. Following the milling steps, the particles were sieved to only retain particle sizes <500 μm.

### Particle Size Distribution:

<90 μm: ~10%
90 μm-125 μm ~20%
125 μm-250 μm ~40%
250 μm-500 μm ~30%

### Example 4

### Particle Production and Sterilization

PVA was modified with pendant norbornene groups for subsequent thiol-ene chemistry according to the present invention. The obtained macromer was termed “PVA-NB”. In a reaction with carbic anhydride in dry DMSO with catalytic amounts of para-toluenesulfonic acid (p-TsOH) PVA-NB was yielded as described in literature (Baudis et al., J. Polymer Sci. A-Polymer Chemistry 54 (2016), 2060-2070). For the present example, the degree of substitution was set with 7.5%.

TABLE 1
Composition of formulations used
for crosslinked PVA particle production

| Formulation | m_{PVA-NB} (g) | m_{GDMA} (g) | mₛₒₗᵥₑₙₜ (g) |
|---|---|---|---|
| 1 | 6.981 | 0.510 | 67.38 |
| 2 | 7.474 | 0.542 | 72.14 |
| 3 | 6.954 | 0.477 | 67.12 |
| 4 | 6.573 | 0.456 | 63.44 |

Hydrogels were produced from PVA-NB with Glycol di(mercapto acetate) (GDMA) as a thiol component for UV-induced crosslinking (i.e. PVA-NB with DS of 7.5% and 50 mol % thiol/ene ratio, see FIG. 2), dehydrated/dried and milled to obtain particles. The material was termed “PVA27-NB7.5-GDMA50”.

Principal network formation reactions and workflow to obtain dry particle materials are outlined in FIG. 1. An aliquot of the obtained particles was treated with γ-radiation for sterilization (measured dose: 27.7-31.2 kGy) in compliance with EN ISO 13485 and ISO 11137.

SEM analysis was performed with a JCM 6000 Benchtop Scanning Electron Microscope manufactured by Jeol Ltd. Particles were sputter coated with gold prior to measurements. Scanning electron microscopy (SEM) was performed with pre- and post-sterilized particles sputter coated with a thin gold layer to investigate the surface morphology. The results are shown in FIG. 3. The sterilization process did not have an influence on the particles' surface morphology. Particle sizes are within a comparable range by visual inspection. Consequences of the milling process are visible at high magnifications (600× and 1000×). Marks caused by the impact blades of the mill are marked by yellow arrows in FIG. 3. All particles show a non-porous surface.

Literature suggests an influence of γ-radiation on the final gel fraction of PVA-based hydrogels. Irradiation of aqueous PVA solutions with high energy radiation (such as γ-radiation) leads to formation of stable hydrogels (Sakurada et al., Mem. Fac. Eng., Kyoto Univ. 21 (1959), 84-93; Park et al., Rad. Phys. Chem. 81 (2012), 857-860). As reported in literature, γ-ray can induce main chain scission (El-Sawy et al., Polymer-Plastics Techn. Engin. 49 (2010), 169-177; Bhat et al., Nucl. Instr. Meth. Physics Res. Section B: Beam Interactions with Materials and Atoms 237 (2005), 585-592). Described effects from the sterilization may impact material properties, such as swelling capacity or degradation. Therefore, the impact of the sterilization step on the material's properties was investigated.

### Example 5

### Swelling Properties

Swelling of pre- and post-sterilized samples was investigated in two different ways, by volumetric swelling (relative volume increase) and mass swelling ratio.

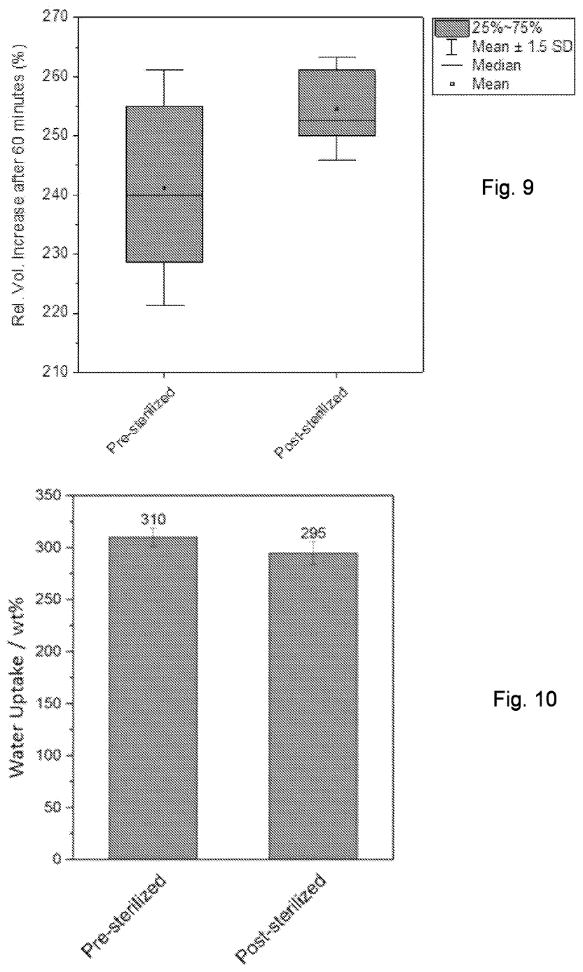

The results are disclosed in FIG. 8; No major differences were detected between pre- and post-sterilized samples. Within the first minutes of swelling, both sample groups showed similar swelling behavior. After approx. 5 minutes, γ-ray sterilized particles exhibited a slightly increased volumetric swelling compared to non-sterilized particles. This trend continued until the end of the observation period. However, found differences in volumetric swelling are small. Statistical significance of found differences was investigated using one-way ANOVA and post-hoc Tukey test. The relative volume increase after 60 minutes of incubation was used for statistical analysis (FIG. 9). Analysis via one-way ANOVA and Tukey test showed no statistically significant difference in the mean values.

The mass swelling ratio results are depicted in FIG. 10. The data obtained showed only minor differences in mass swelling behavior. Particle sterilization has only minor impact on mass swelling. For the sterilized samples a slightly lower mass swelling ratio (MSR) was observed.

Results of the swelling assays did not reveal any statistically significant differences in the swelling behavior. Investigation of volumetric swelling showed that, especially within the first minutes of swelling, behavior of both, pre- and post-sterilized samples, was within a similar range.

### Example 6

### Study on In Vitro Degradability

This example describes the degradation behavior of crosslinked PVA particles. Sterilization was carried out for “PVA-NB7.5-GDMA50” particles (γ-dose: 27.7-31.2 kGy, EN ISO 13485 and ISO 11137). For this study, particles (pre- and post-sterilization by γ-irradiation) were filled into vials and immersed in PBS at 37 °C. Mass loss was determined weekly over 5 weeks after washing with deionized water and lyophilization.

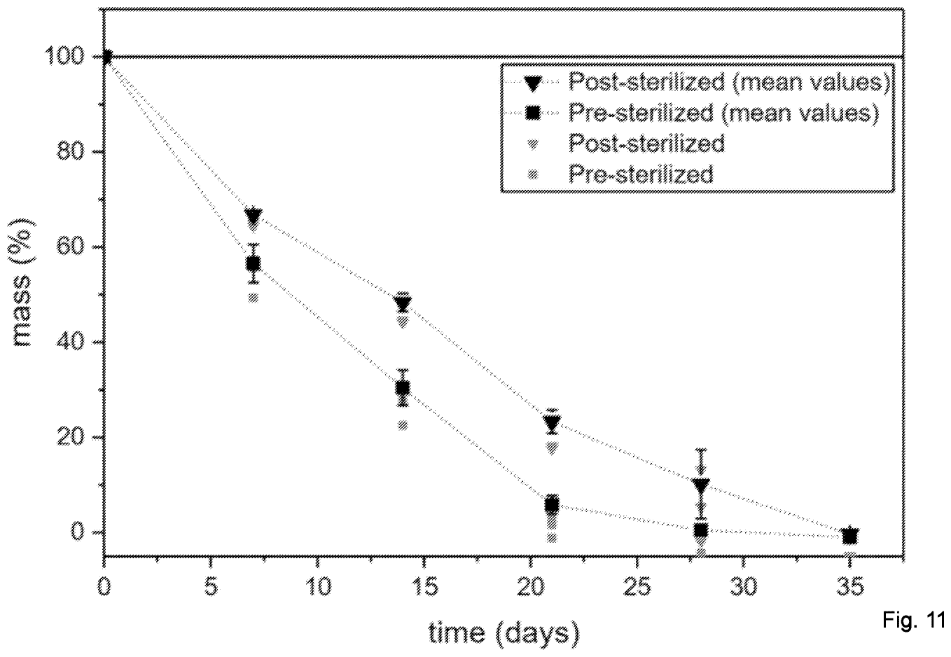

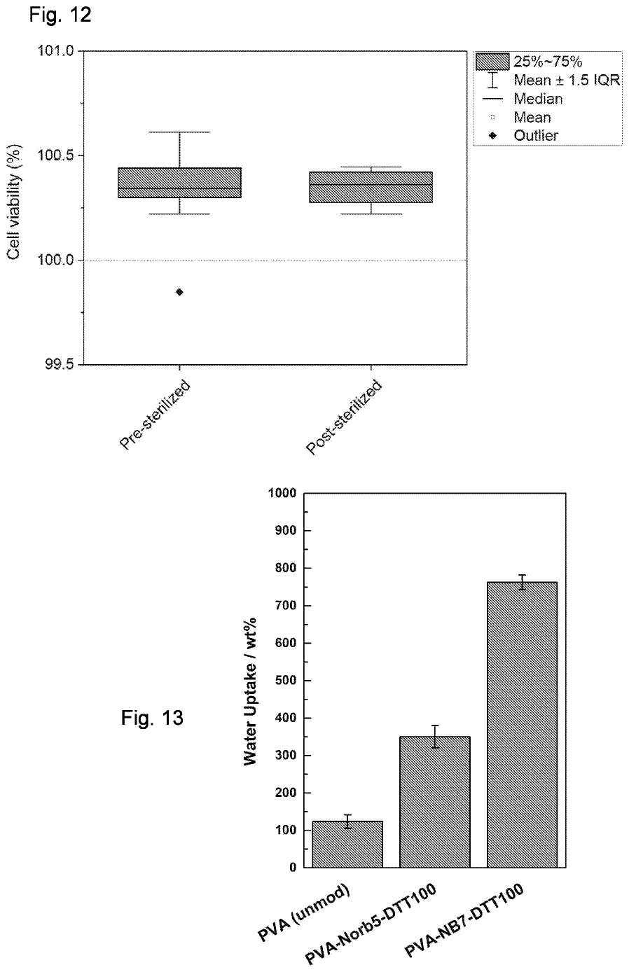

Both samples (i.e. pre-sterilization and post-sterilization particles) showed good degradation behavior over the investigated time period. Full degradation was noted after 5 weeks for both groups. Degradation results (mass loss in % over time) are shown in FIG. 11.

### Example 7

### Cytotoxicity

Cytotoxicity of pre- and post-sterilized samples was assessed using a BrdU Cell Proliferation ELISA (enzyme-linked immunosorbent assay) Kit (colorimetric) (ab126556) obtained from Abcam plc. BrdU is a synthetic nucleoside analogue and was used for monitoring cell proliferation via incorporation of said molecule into the cell's DNA. The assay was performed according to the instructions of the manufacturer as described below.

MG63 cells were seeded in DMEM High Glucose 5% FCS, 1% L-Glu, 1% P/S and allowed to adhere for 6 h. Both sample groups (pre- and post-sterilized) were UV sterilized in advance and were investigated in 16 replicates each. The control group (no sample) was investigated in 8 replicates. After cell adherence, a defined amount of sample granules was suspended in a defined amount of DMEM and added to the respective wells to reach a concentration of 10 mg/mL. Subsequently, BrdU reagent was added and allowed to incorporate for 24 h. During the incubation time, BrdU was incorporated into the proliferating cells' DNA. Cell cultures with high proliferation rates therefore incorporate more BrdU into their DNA. Following incubation, sample granules were removed, cells were fixed, and the DNA was denatured.

Antibodies were added subsequently and were allowed to attach to respective sites. Anti-BrdU monoclonal Detector antibody (antibody 1) selectively adhered to BrdU incorporated in the DNA. After a washing step to remove unbound antibodies, Peroxidase Goat Anti-Mouse IgG Conjugate (antibody 2) was added and selectively bound to antibody 1. Antibody 2 contained horseradish peroxidase (HRP).

Unbound antibody was washed away after incubation. TMB Peroxidase Substrate was added and allowed to react for 30 minutes. During this time, reaction B) took place and a blue colored product was formed. In the end, a stop solution was added to obtain a yellow product, which was then quantified via UV absorption measurement (450 nm and 550 nm).

Samples were tested in 16 replicates, control groups in 8 replicates (cells+medium only), and background measurements (no cells, only reagents) in 8 replicates.

To receive data with a minimum amount of distortion, two absorbance values were measured (450 nm and 550 nm). Cell viability was calculated by correction of absorbance values of the sample (450 nm) with absorbance values at 550 nm and background absorbance. The corrected values were used in the following equation.

$$Cell viability ( \% ) = \frac{{Absorbance}_{Sample}}{{Absorbance}_{Control}} * 100$$

Statistical analysis was carried out in the software Origin Pro 2016G. One-way ANOVA and Dunn-Šidak multiple comparison were carried out with a significance level of 0.05.

The results are depicted in FIG. 13. Both investigated groups did not show any signs of deteriorated cell viability during 24 h of contact with the samples.

For statistical analysis one-way ANOVA and Dunn-Šidak multiple comparison were performed. Both tests showed no statistically significant differences in the mean values of pre- and post-sterilized particles. Obtained data did not imply negative effects of crosslinked PVA particles on cell proliferation. Furthermore, no differences in the effects of pre- and post-sterilized samples were found.

### Example 8

### Absorption Capabilities

To characterize a family of poly(vinyl alcohol)-based networks exploiting thiol-norbornene step-growth mechanism a set of PVA-norbornene modifications are possible within the present invention. Firstly, the variation of the degree of norbornene-group substitution along the linear polymer backbone before crosslinking was investigated. Secondly, an additional type of norbornene modification was tested. For previous material generations PVA was modified by reaction with carbic anhydride to obtain a linear PVA backbone modified with anionic-charged norbornene groups due to the sodium carboxylate functionality.

To investigate the influence of the backbone charge on the materials' swelling properties, a non-ionic variant was also investigated. Therefore 5-norbornene-2-carboxylic acid was used for modification of PVA instead of carbic anhydride to produce “PVA-Norb”, the corresponding non-ionic variant of PVA-NB (both synthetic strategies are depicted in FIG. 6).

PVA was modified with pendant norbornene groups for subsequent thiol-ene chemistry. The obtained macromer was termed “PVA-NB” (see FIG. 6, reaction path A). In a reaction with carbic anhydride in dry DMSO with catalytic amounts of para-toluenesulfonic (p-TsOH) acid PVA-NB was yielded as described in literature (Baudis et al., J. Polym. Sci. Part A: Polym. Chem. 54 (2016), 2060-2070). The achieved degree of substitution of PVA could be regulated by according changes to the used amount of carbic anhydride (i.e. the norbornene group precursor used for the modification reaction) for the reaction.

Additionally, PVA-Norb was synthesized as non-ionic variant. The obtained macromer was termed “PVA-Norb” (see FIG. 6, reaction path B). The strategy was to create a reactive intermediate of 5-norbornene-2-carboxylic acid (i.e. carboxylic anhydride) that can be isolated for further transformations. Firstly, a carboxylic anhydride was generated from 5-norbornene-2-carboxylic acid (i.e. 5-norbornene-2-carboxylic anhydride) using EDC-coupling chemistry in DCM as solvent.

The isolated anhydride was then used for modification of PVA in DMSO to yield PVA-Norb after dialysis and lyophilization with a degree of substitution of 5% of repeating units of PVA being modified with a pendant non-ionic norbornene group.

Absorption capabilities as central characteristic of passive hemostatic materials were used as a metric to investigate the influence of the type of PVA modification. Prototype particle materials were produced and swelling behavior was quantified.

Hydrogels were produced from PVA-Norb and PVA-NB each with an equimolar amount of DTT for UV-induced crosslinking (i.e. “PVA-NB7-DTT100, “PVA-Norb5-DTT100”), dehydrated/dried and milled to obtain particles.

TABLE 2
Formulations for hydrogel preparations

| | | PI stock | | PI stock |
|---|---|---|---|---|
| | PVA | for PVA | Dithiol | for thiol |
| Formulation | Macromer | solution | crosslinker | solution |
| PVA- | 3000 mg | 26.720 | DTT | 2.796 |
| NB7- | (~3.626 | mL | 307.6 mg | mL |
| DTT100 | mmol | | 1.994 | |
| | norbornene) | | mmol | |
| PVA- | 3000 mg | 26.773 | DTT | 2.273 |
| Norb5- | (~2.947 | mL | 227.3 mg | mL |
| DTT100 | mmol | | 1.474 | |
| | norbornene) | | mmol | |

Additionally, non-modified PVA (“PVA (unmod)”) was used as reference material to prove the effect of polymer modification and crosslinking on material hydrophilicity and absorption properties. For the reference particle material group of unmodified PVA, no hydrogel was synthesized and no prior PVA modification was necessary. Flakes of PVA (Sigma Aldrich, 27 kDa, 98% degree of hydrolysis) were directly subjected to milling and particle size fractionation.

### Particle Milling and Size Fractionation

The crosslinked and dried PVA-based matrices were milled using a mill with water-cooled milling compartment (A10 basic, IKA, milling intervals were set to 30 s per run). After milling, particles were fractionated according to their sizes using steel mesh sieves with various mesh sizes (Retsch, sizes 500 μm, 300 μm, 200 μm, 100 μm).

To investigate the influence of different particle sizes the following particle size fractions were used:
Small particles: 100-200 μm
Medium particles: 200-300 μm
Large particles: 300-500 μm
Mixed particles: mix of small, medium, and large particles (1:1:1 by weight).

If not stated otherwise, the particle size mix was used for all swelling evaluations.

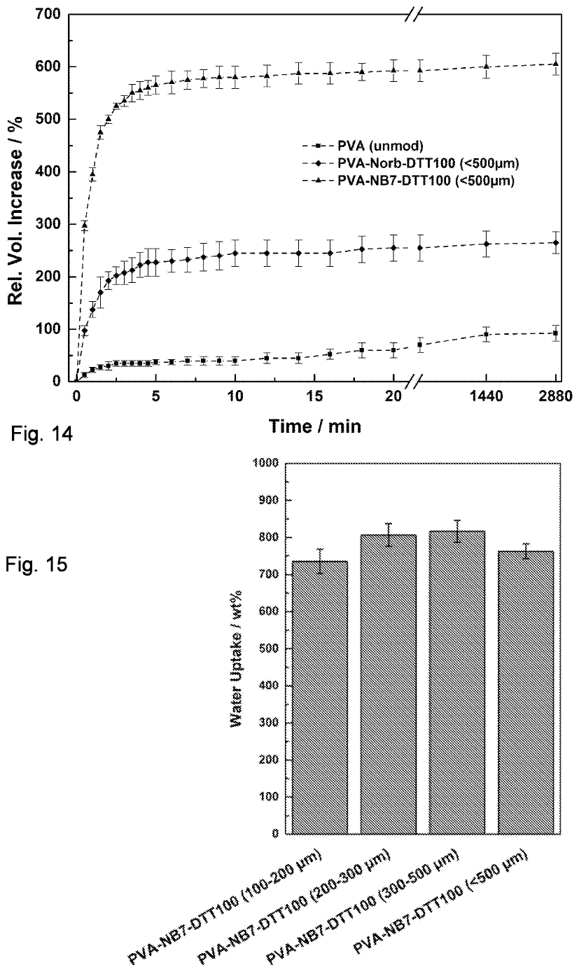

The results are depicted in FIG. 13 and FIG. 14.

Particles from modified PVA showed much higher gravimetric swelling compared to unmodified PVA. The ionic PVA modification variant PVA-NB (“PVA-NB7-DTT100”) showed more than double the water uptake of the non-ionic variation (PVA-Norb, “PVA-Norb5-DTT100”). The ionic character of the modification leads to a more hydrophilic network that can take up more water into the matrix.

For volumetric swelling (relative volume increase) similar effects can be determined as for the gravimetric experiment (water uptake). Modified PVA particles (i.e. “PVA-Norb5-DTT100” and “PVA-NB7-DTT100”)) showed pronounced volumetric swelling during the first minutes of the experiment after which a plateau was reached. Only minor swelling takes place for unmodified PVA during the first minutes.

While for the other groups a plateau was reached after the first minutes of swelling, unmodified PVA showed relatively high volumetric increase at the 24 h mark. Unmodified PVA exhibited a more sustained swelling behavior over longer periods of time (i.e. a notable volumetric increase at 24 h of swelling).

Maximum relative volume increase of PVA-NB and PVA-Norb were around 600% and 350%, respectively. Like the gravimetric swelling results, PVA-NB-based particles showed the most pronounced swelling due to the ionic modification of the network. Additionally, PVA-NB-based particles showed faster swelling kinetics, reaching the plateau of swelling after around the same swelling time as PVA-Norb-based particles (i.e. 5-10 minutes).

### Example 9

### Particle Production and Particle Size Distribution

A hydrogel was created (PVA-NB with DS of 7% and a molar DTT amount to achieve a 100 mol % thiol/ene ratio, i.e. “PVA-NB7-DTT100”), dehydrated/dried and milled to obtain particles.

TABLE 3
Formulations for hydrogel preparations

| | | PI stock | | PI stock |
|---|---|---|---|---|
| | PVA | for PVA | Dithiol | for thiol |
| Formulation | Macromer | solution | crosslinker | solution |
| PVA- | 3000 mg | 26.720 | DTT | 2.796 |
| NB7- | (~3.626 | mL | 307.6 mg | mL |
| DTT100 | mmol | | 1.994 | |
| | norbornene) | | mmol | |

These particles were used to evaluate the influence of particle size on swelling behavior. The particles were fractionated according to particle diameter by sieving through steel mesh sieves with different mesh sizes to produce the following size fractions of particles:
Small particle sizes (diameters 100-200 μm)
Medium particle sizes (diameters 200-300 μm)
Large particle sizes (diameters 300-500 μm)
Mixed particle sizes (diameters <500 μm)

The swelling of these particle materials in PBS was investigated and quantified by a volumetric and a gravimetric method to evaluate the influence of particle size on the swelling characteristics.

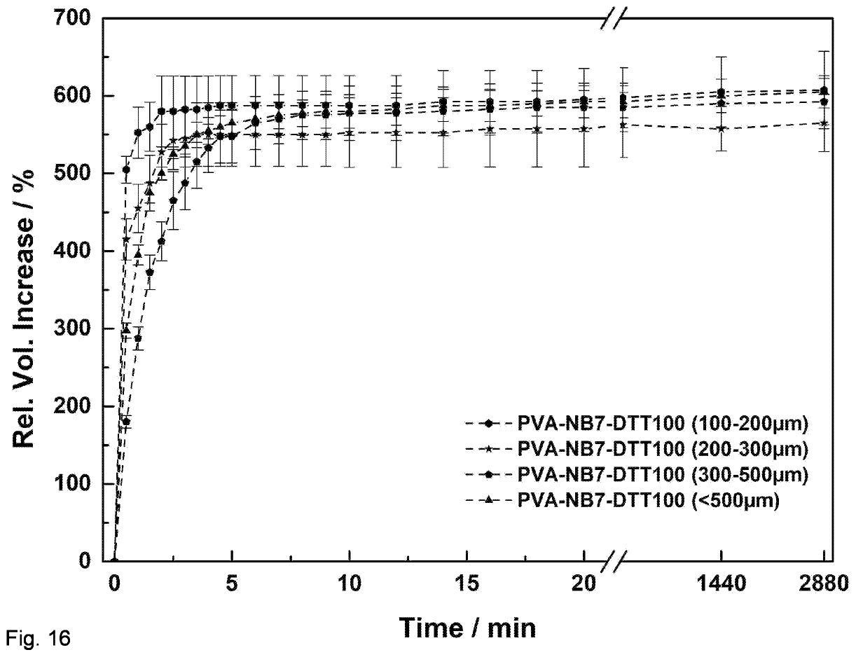

The results are depicted in FIG. 15. Largest differences in water uptake between the tested groups are less than 10%. This is due to the fact that all tested particle fractions are based on the same matrix material and volumetric swelling experiments show that equilibrium swelling is already reached at 24 h, i.e. the time point at which gravimetric swelling is evaluated. Therefore, this result confirms that the change of particle sizes does not alter the equilibrium swelling since the chemical identity and network architecture of the underlying polymer is unchanged.

Particle swelling is especially pronounced during the first minutes of contact with the aqueous phase (FIG. 16). The particle phases reach their maximum relative volume increase after 5-10 minutes and thereafter the volume increase reaches a plateau. These principal swelling characteristics can be observed for all particle size groups. There are only minor differences in swelling behavior between the groups. After reaching the plateau of swelling, no large differences between the particle sizes can be found with relative volume increases of around 550-600% and water uptakes of roughly 700-800 wt % after 24 h.

### Example 10

### Alternative Crosslinkers and Crosslinking Procedure

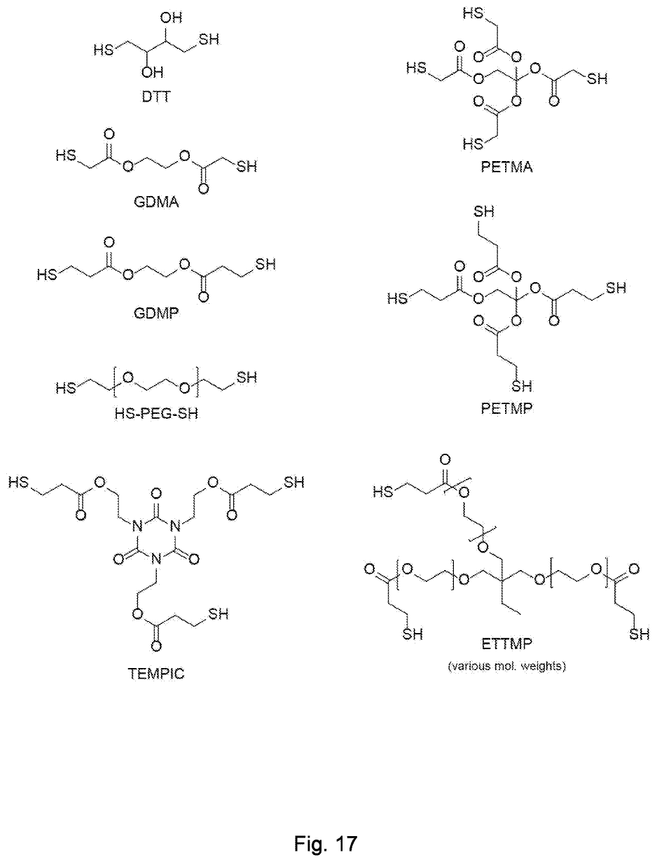

A range of multifunctional thiol-compounds can be used for thiol-ene crosslinking of norbornene systems. A list of alternative thiols for thiol-ene hydrogels is provided with examples from literature (FIG. 17):
PVA-NB was crosslinked with DTT (dithiothreitol) for 3D-printing and hemostatic materials.
Thiol-functionalized PEG can be used for thiol-ene chemistry. Dithiol-functionalized poly(ethylene glycol) of average molecular weight of 2000 Da (HS-PEG-SH) was used for crosslinking of PVA-NB to produce blends with gelatin for hemostatic applications. Tetrafunctional thiolated PEG-compounds have been utilized. Shorter ethylene glycol spacers are also possible for thiol-ene chemistry.
A wide range of multifunctional thiol crosslinkers based on mercaptopropionic acid and mercaptoacetic acid and their esters are usable for thiol-ene chemistry. This includes bifunctional crosslinkers (e.g. glycoldimercapto propionate GDMP and glycoldimercapto acetate GDMA) as well as a wide range of multifunctional thiols.
Thiol-functionalized polyglycidols
Mercaptophenylacetic acid-based crosslinker
Thiolated polymers like PVA or gelatin. But also thiolations of other polymers are possible (e.g. collagen, dextran, chitosan, heparin).
Crosslinkers based on cysteine as reactive thiol (peptides, polymers)
Proteins with free thiols.

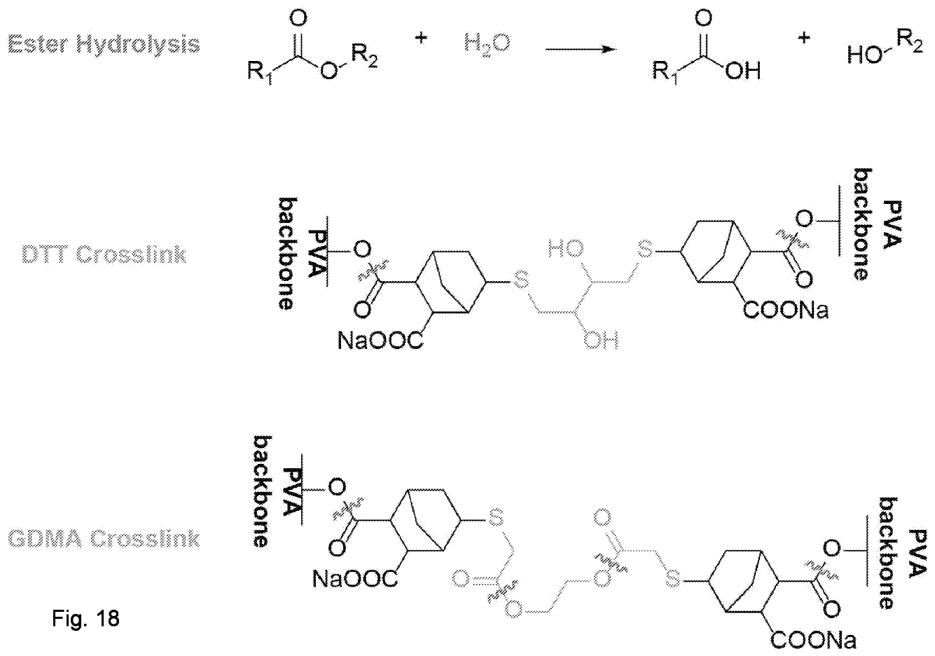

The degradability of PVA-NB networks relies largely on ester hydrolysis since the PVA backbone is not degradable under relevant physiological conditions. Therefore, experiments were conducted with DTT and GDMA as model thiol crosslinkers. DTT is a highly hydrophilic molecule enabling materials with good water uptake and hemostatic capabilities as passive hemostats. GDMA was chosen due to its increased degradability in PVA-NB backbones, as demonstrated in preliminary experiments. GDMA-crosslinked materials degrade faster than DTT because two additional moieties are included per crosslinking moiety in the network, effectively doubling the amount of ester bonds per crosslink and increasing the chance for hydrolytic breakage of the crosslink. This is also illustrated in FIG. 18.

Then the influence of thiol-ene ratio and degree of substitution during crosslinking was investigated. The network density of an idealized step-growth network based on PVA-NB is based on molecular weight of PVA, the degree of substitution and the type and amount of thiol crosslinker. The molecular weight of PVA (27 kDa) and the type of thiol crosslinker (DTT) were fixed for this study to investigate the influence of the amount of crosslinker based on the resulting thiol-norbornene ratio and the degree of substitution. More specifically, three model particle systems based on PVA-NB were investigated:
Low DS and low thiol-norbornene ratio (i.e. 7% DS and 50% SH/ene)
Low DS and high thiol-norbornene ratio (i.e. 7% DS and 100% SH/ene)
High DS and low thiol-norbornene ratio (i.e. 14% DS and 50% SH/ene)

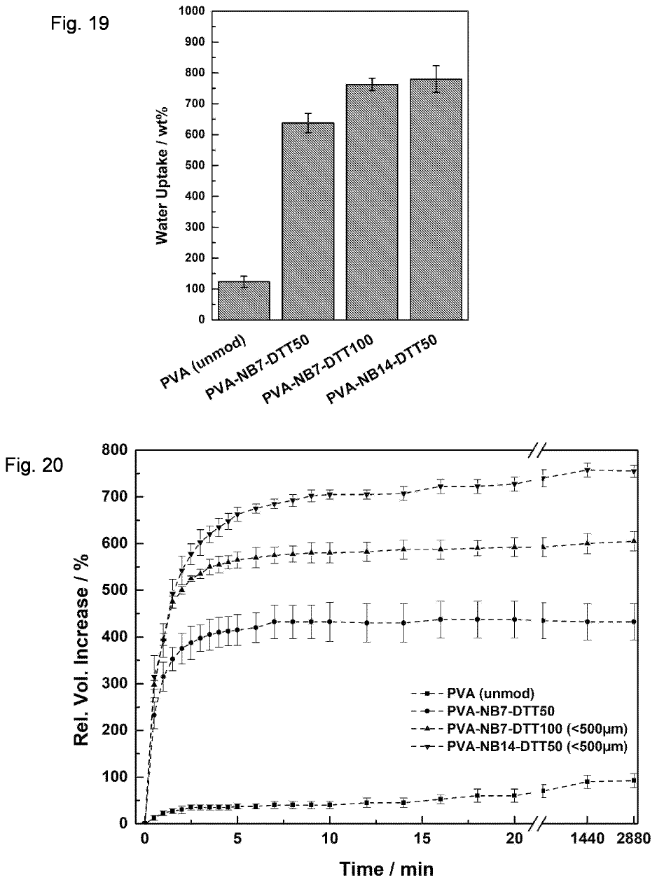

The comparison of particles of low DS with high thiol content and high DS with low thiol content is of particular interest to investigate the influence of DS and thiol/ene as parameters. Produced particles were investigated for their gravimetric and volumetric swelling characteristics in PBS at room temperature (FIGS. 19 and 20).

TABLE 4
Formulations for hydrogel preparations

| | | PI stock | | PI stock |
|---|---|---|---|---|
| | PVA | for PVA | Dithiol | for thiol |
| Formulation | Macromer | solution | crosslinker | solution |
| PVA-NB7- | 3000 mg | 26.860 | DTT | 1.398 |
| DTT50 | (~3.626 | mL | 139.8 mg | mL |
| | mmol | | 0.906 | |
| | norbornene) | | mmol | |
| PVA-NB7- | 3000 mg | 26.720 | DTT | 2.796 |
| DTT100 | (~3.626 | mL | 307.6 mg | mL |
| | mmol | | 1.994 | |
| | norbornene) | | mmol | |
| PVA-NB14- | 3000 mg | 26.772 | DTT | 2.283 |
| DTT50 | (~5.920 | mL | 228.3 mg | mL |
| | mmol | | 1.480 | |
| | norbornene) | | mmol | |
| PVA-NB7- | 3000 mg | 26.809 | GDMA | 1.906 |
| GDMA50 | (~3.626 | mL | 190.6 mg | mL |
| | mmol | | 1.236 | |
| | norbornene) | | mmol | |
| PVA-NB7- | 3000 mg | 26.619 | GDMA | 3.812 |
| GDMA100 | (~3.626 | mL | 381.2 mg | mL |
| | mmol | | 2.471 | |
| | norbornene) | | mmol | |

For gravimetric swelling assessment, unmodified PVA particles were used as a reference material with minor water uptake capabilities when compared to the PVA-NB/DTT crosslinked system. Particles of low DS and low crosslinker content (“PVA7-DTT50”) showed a decreased water uptake capability when compared to the other two tested modified particle groups. The difference in measured water uptake between low DS/high crosslinker content and high DS/low crosslinker content (“PVA-NB7-DTT100” versus “PVA-NB14-DTT50”) is not statistically significant (at the 0.05 level). Both material groups are based on a similar molar amount of crosslinks throughout the network, explaining the similarities.

Relative volumetric increase of the particle phase followed the aforementioned trend for the modified particle materials (FIG. 20): Swelling slowed down after 5-10 minutes and a plateau was reached. However, the group “PVA-NB14-DTT50” showed some further increases at later time points from around 700% to roughly 750% after 24 h of swelling.

The groups “PVA-NB7DTT50” and “PVA-NB7-DTT100” showed a somewhat lower volumetric swelling of around 400% and 550%, respectively.

The slope of initial relative volume increase as measure of swelling kinetics shows similarly fast initial swelling of all three groups of modified PVA-NB particles.

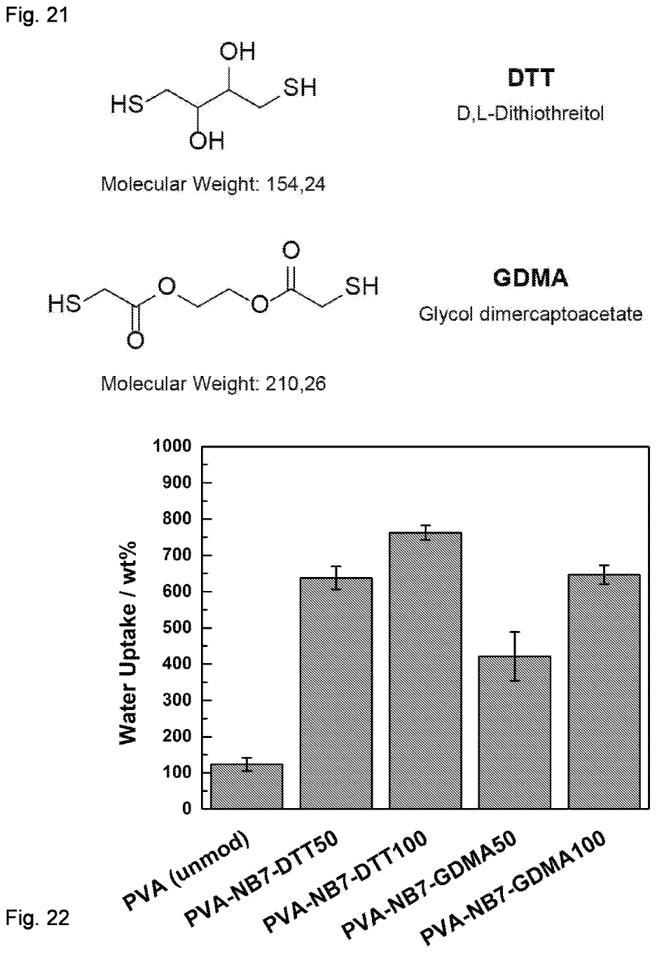

Then, the influence of thiol crosslinkers during crosslinking was investigated: GDMA was investigated as an alternative dithiol compound for crosslinking of PVA-NB (DS of 7%). Structures are shown in FIGS. 17 and 21. Thiol-ene hydrogels from PVA-NB7-DTT and PVA-NB7-GDMA were created with varying thiol-norbornene ratios of 50 mol % and 100 mol %.

Produced hydrogels were dehydrated/dried and milled to yield particles. Unmodified PVA was used as a non-crosslinked and non-modified reference material. The influence of these changes in network composition on swelling characteristics was investigated by gravimetric water uptake measurements as well as relative volume increase measurements of the particle phase during swelling in an excess of PBS at room temperature (FIG. 22).

PVA-NB materials crosslinked with GDMA showed a lower water uptake capability compared to their DTT-crosslinked counterparts. This can be explained by the fact that DTT is more hydrophilic than GDMA. While DTT is readily soluble in water, hydrogel precursor formulations based on GDMA as crosslinker must contain ethanol to be able to solubilize GDMA as it is not fully soluble in water. Furthermore, higher crosslinker contents lead to higher water uptake. This could again be explained by added crosslinker moieties restricting the efficient H-bonding of the PVA backbone.

The chemically modified PVA-NB particles overall demonstrated high swellability.

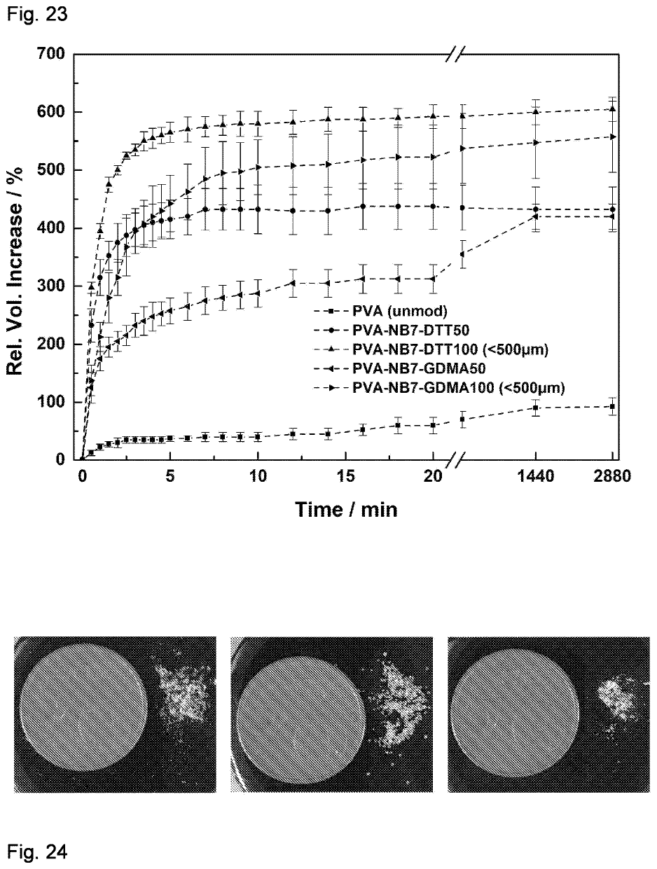

Volumetric swelling experiments showed fast swelling of all chemically modified and crosslinked particle groups (FIG. 23). However, relative volume increase of GDMA-crosslinked materials was slower than for the DTT-crosslinked counterparts. This could be explained by hydrophilicity differences between the two crosslinkers. Furthermore, GDMA-crosslinked particles showed also swelling towards later time points. Especially the relative volume increase of the group “PVA-NB7-GDMA50” between the 20 and 60 min marks and between the 60 min and 24 h mark are notable, showing increases of roughly 300-350% and 350-400%, respectively. This delayed swelling effect is due to the decrease in network polarity which effects slower water-uptake over a longer time.

Particle groups reach the similar final relative volume increases when crosslinked with the same molar amount of crosslinker (i.e. 50% DTT versus 50% GDMA and 100% DTT versus 100% GDMA) at roughly 400% and between 500-600%, respectively. This is due to the fact that the respective groups contain a similar amount of crosslinks throughout the network.

Concluding, it was shown that unmodified PVA was found to provide only delayed and minor swelling and water uptake, making it less suitable as a passive hemostatic material. Also, PVA modified with non-ionic norbornene groups (i.e. “PVA-Norb”) provided distinctly less pronounced swelling capabilities, both gravimetric and volumetric when compared to its ionically modified counterpart PVA-NB.

Overall, all tested variations of chemically crosslinked PVA-NB particles showed a high capacity for water uptake. However, for PVA-NB increased swelling performance can be stated when compared to PVA-Norb, making it a superior material variation suitable for further in vivo testing.

### Example 11

### In Vivo Efficacy Testing

PVA-NB crosslinked with GDMA was tested in vivo as dry particles and the size of the particles was varied, and three particle size groups were defined (FIG. 24).

More specifically, PVA-NB with a MW of 27 kDa and a DS of 7.5% was synthesized, crosslinked with GDMA, dried, milled, and tested in vivo as dry particles. As solvent for the formulation a mixture of ethanol and deionized water in a ratio of 2:1 was used with a PI (Irgacure 12959, α-hydroxy-4-(2-hydroxyethoxy)-α-methylpropiophenon) concentration of 0.25 wt %. Detailed composition of the formulation is given in the following table.

TABLE 5
Composition of formulation for
particles used for in vivo testing

| Formulation | m_{PVA-NB} (g) | m_{GDMA} (g) | mₛₒₗᵥₑₙₜ (g) |
|---|---|---|---|
| 1 | 6.981 | 0.510 | 67.38 |
| 2 | 7.474 | 0.542 | 72.14 |
| 3 | 6.954 | 0.477 | 67.12 |
| 4 | 6.573 | 0.456 | 63.44 |

Particles were sieved into following fractions:
Small particles: <200 μm (“small”)
Large particles: 200-500 μm (“large”)
Mixed particles: <500 μm (“mixed”)

For the mixed fraction, all particles directly from the mill smaller than 500 μm were used (no mixing step as described above).

Each group was tested in triplicate. In addition to that, particles were also applied as a slurry (thrombin solution in saline, 1000 units/mL).

A porcine liver biopsy punch model (10 mm biopsy punch) was used in a non-heparinized pig for this study. Initial bleedings were assessed by application of pre-weighted gauze for six seconds and subsequent weighing. After applying the dry hemostatic particles (500 mg per application), they were approximated manually with a piece of dry gauze for two minutes. Afterwards, the gauze was removed cautiously.

Time to hemostasis (TTH) was assessed visually.

In addition to that, particles were also applied as a slurry (thrombin solution in saline, 1000 units/mL).

A porcine liver biopsy punch model (10 mm biopsy punch) was used in a non-heparinized pig for this study. Initial bleedings and time to hemostasis (TTH) were assessed. Results are summarized in the following table and are discussed below:

TABLE 6
Results of in vivo efficacy assessment

| | | Initial | | | | | |
|---|---|---|---|---|---|---|---|
| | | Bleeding | | | TTH | | |
| Application | # | (g/min) | Mean | SD | (min) | Mean | SD |
| Large Particles | 1 | 3.1 | 5.43 | 2.17 | 7 | 7.00 | 1.00 |
| (200-500 μm) | 2 | 5.8 | | | 6 | | |
| | 3 | 7.4 | | | 8 | | |
| Small Particles | 1 | 1.6 | 2.83 | 1.80 | 2 | 4.92 | 3.00 |
| (<200 μm) | 2 | 2 | | | 4.75 | | |
| | 3 | 4.9 | | | 8 | | |
| Mixed Particles | 1 | 3.8 | 4.10 | 0.44 | 2 | 2.67 | 1.15 |
| (<500 μm) | 2 | 3.9 | | | 2 | | |
| | 3 | 4.6 | | | 4 | | |
| Slurry Application | 1 | 6.4 | — | — | 7.5 | — | — |

The obtained data show that the hemostatic efficacy is dependent on the particle size distribution. Large particles showed low to no adhesion to gauze, which was beneficial for the applicability. However, hemostatic efficacy was not satisfactory.

Small particles did show adhesion to dry gauze. This led to difficulties when removing the gauze from the wound site. Furthermore, particles were packed denser due to their small size, and with this the gaps between the particles were smaller. The higher surface-to-volume ratio caused more rapid swelling (as shown in FIG. 16). It is suspected that the combination of the described effects led to formation of a gelled barrier layer which hindered blood to penetrate the particle layer further. As a result, non-clotted blood oozed from the edges and cracks, respectively, of the particle layer.

Mixed particles showed the best results. They combine the advantages of large particles (enabling penetration of blood into the particle layer) and small particles (quick swelling due to high surface-to-volume ratio).

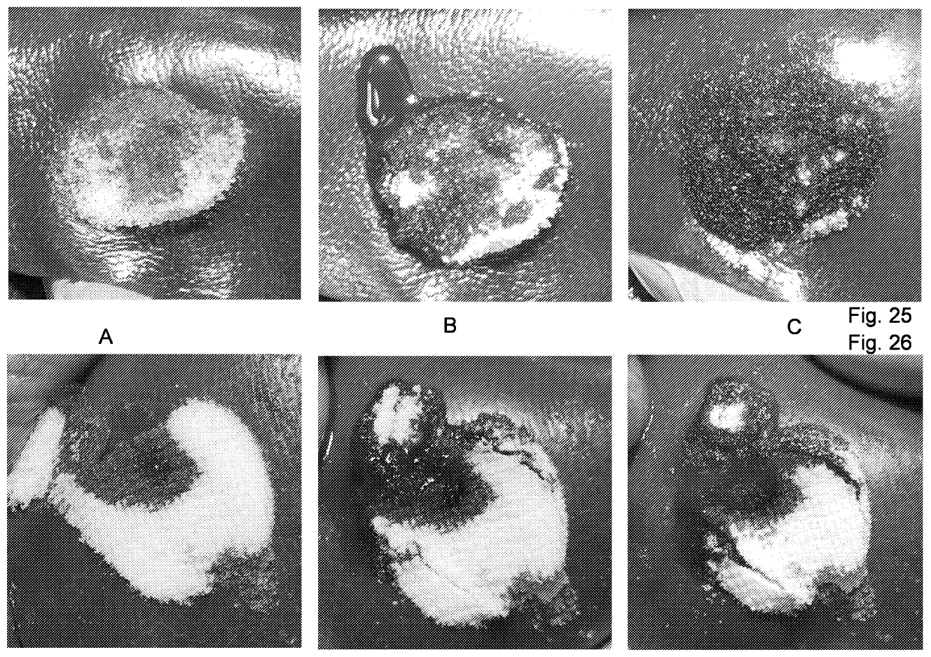

The results in detail (FIGS. 25 to 28):

### Large Particles (200-500 μm; FIG. 25)

After two minutes of manual fixation, the gauze used for approximation could be removed easily due to the low adhesion of the particles to gauze. Hemostasis was not achieved after two minutes. In all tests oozing bleedings occurred (see FIG. 25 B). This was most likely caused by large gaps between the particles and the low material surface area compared to smaller particles (the smaller a particle, the higher the surface-to-volume ratio).

As shown in Table 6, TTH values, even for low bleedings, were relatively high. The minimum TTH found in this study was six minutes with an initial bleeding of 5.8 g/min.

### Small Particles (<200 μm; FIG. 26)

Although good flowability of the particles (no particle clumping, good pourability) was a characteristic of this particle size, the application was associated with problems. After manual approximation, removing gauze from the wound site was problematic due to high adhesion of the material to the gauze and low adhesion of the material to the wound.

Five samples in total were used for this study, but two tests were discarded because most of the applied particles were removed from the wound site with the removal of gauze, which led to rebleeding of the wound. High adhesion to gauze also led to cracks in the formed particle layer after gauze removal (see FIG. 26A). It should be noted that the initial bleeding rates used for these tests turned out to be lower compared to the other sample groups.

However, with three tests hemostasis was achieved.

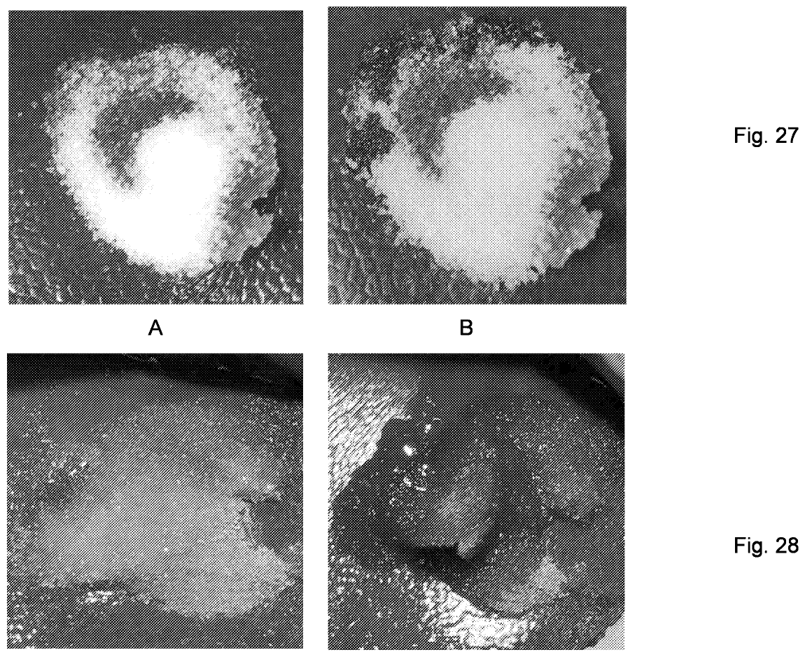

### Mixed Particles (<500 μm; FIG. 27)

Removal of gauze after application was not problematic in this case. Similar to the particle fractions tested before, good flowability was also given here. Hemostasis was achieved within 2 to 4 minutes, faster than for large or small particles.

### Slurry Application (FIG. 28)

For slurry application, particles of small and large fractions were mixed in a 1:1 mass ratio. A total mass of 1 g was filled into a syringe of a FLOSEAL® syringe mixing device, and the other syringe was filled with 1 mL of thrombin solution (1000 units/mL).

Preparation and application of the slurry were carried out similarly to FLOSEAL® hemostatic matrix. Approx. half of the prepared slurry was applied to a wound with an initial bleeding of 6.4 g/min. TTH was 7.5 minutes. A second application with the same slurry was not possible due to its increase in viscosity during ten minutes hold time between applications. Most likely, this was caused by further swelling during this time period.

This preliminary test showed that slurry preparation and application is possible in general, but dependent on numerous factors such as particle size distribution, amount of used particles, amount of thrombin solution, the concentration of thrombin in solution and swelling time of PVA-NB-GDMA particles in thrombin solution.

### Example 12

### Comparative Results of the PVA Particles According to the Present Invention Compared to the Porous PVA Preparations Disclosed in WO 03/063922 A1

Porous PVA preparations as disclosed in WO 03/063922 A1 were prepared as follows:

A 20% w/v 27 kDa PVA solution was produced by weighing PVA in deionized water, heating up to 60 °C and stirring for 2 h. To produce freeze/thawed samples (PF 2 27 kDa), the solution was frozen for 16 h, followed by thawing for 4 h at 5 °C and 4 h at room temperature. This cycle was repeated 9 more times, followed by lyophilization. To produce foamed samples (PF1 27 kDa), the solution was diluted to 14% (w/v) and foamed with the addition of 5% (w/v) of Triton X-100, by stirring at 1000 rpm for one minute. The sample was then shock-frozen at −78 °C and lyophilized.

PVA-NB-DTT samples were created by crosslinking PVA-NB of DS of 7% with DTT to achieve an >100 mol % thiol/ene ratio (“PVA-NB7-DTT127”). For the “oven dried” samples, the material was dehydrated and dried at 60 °C. For the “lyophilized” samples, the material was reconditioned, frozen and lyophilized to preserve porosity.

These preparations obtained were compared to the PVA particles according to the present invention with respect to their porosity via nano-CT measurements

The porosity of physically crosslinked materials (PVA-PF1 27 kDa, PVA-PF2 27 kDa) as well as thiol-NB-crosslinked materials was compared by nano-computed tomography (Nano-CT) analysis. For the measurements, samples were taken of the bulk material (before the milling step was performed).

Measurements were carried out on Scanco μCT 50 and porosity was evaluated by intensity-based density calculation, based on the correlation of porosity and attenuation in CT (similar to the methods reported by Withjack et al. (SPE Formation Evaluation, 3 (1988), 696-704; doi: 10.2118/16951-PA), Taud et al. (J. Petrol. Sci. Engin. 47 (2005), 209-217) and Bruns et al., Adv. Water Resour. 107 (2017), 32-42)). The resulting scans were analyzed based on a method based on the correlation between porosity and attenuation and intensity in CT, to give the proportion of non-filled volume to filled solid volume.

The results as shown in FIGS. 30 and 31. are given as the proportion of non-filled volume to filled solid volume and serve as a quantification of the material's porosity. As shown in FIG. 30, the physically crosslinked PVA-based materials, “PF1 27 kDa foamed” and “PF2 27 kDa freeze/thawed”, showed a visibly porous structure. In contrast, “PVA-NB7-DTT172 oven-dried” did not show any pores in the nano-CT. Swelling in this material thus was confirmed to result purely from the ability of the covalent network to absorb water, rather than from porosity.

The method of drying was further shown to impact the porosity, as depicted in the CT scan of the norbornene-thiol crosslinked PVA material, which was frozen and lyophilized after crosslinking (“PVA-NB7-DTT172 lyophilized”).

The intensity-based calculation of solid volume per overall volume depicted in FIG. 31 confirms the trends observed in the CT images. The mode of drying largely impacts the structure of the material. As expected, the material that was dried slowly in the oven showed no pores, whereas the PVA-NB-DTT material that was frozen and lyophilized only had ~11% solid share.

For the physically crosslinked materials, the foamed material PF1 showed the lowest solid share of 9%. The freeze/thawed material PF2 showed 31% solid share. The foamed material showed larger pores, which are prone to be destroyed during the milling step, whereas the freeze/thawed material showed a more even porous structure that allowed for pores also within the milled particles.

### Materials and Methods

### Synthesis of Various Modifications of PVA-Norbornene

### Synthesis of PVA-NB

Herein, the procedure is described to obtain PVA-NB with a degree of substitution of 7% (in relation to the repeating units in PVA as determined with proton NMR spectroscopy).

For PVA-NB with 14% degree of substitution the amount of carbic anhydride was doubled.

The synthesis was carried out under Argon atmosphere. PVA (10 g, 27 kDa, 98% degree of hydrolysis) and p-toluenesulfonic acid (20 mg, 0.12 mmol) were dissolved in 200 mL of DMSO (dimethyl sulfoxide, anhydrous) at 70 °C. Temperature was then decreased and kept at 60 °C. The modification reaction was started by adding a solution of carbic anhydride (2.82 g, 17.2 mmol) in 50 mL DMSO to the stirred PVA solution. The obtained solution was stirred for 16 h at 60 °C under Argon atmosphere. The product-containing solution was then dialyzed against 10 mM sodium bicarbonate solution and deionized water with multiple changes for at least 48 h. Lyophilization of the dialyzed solution yields the polymer as white solid (~95% yield). 1H-NMR (400 MHz, D₂O): 6.27 (NB CH═CH), 4.20-3.70 (PVA CH), 3.34 (NB CH), 3.13 (NB CH), 2.13 (PVA-acetyl CH₃), 1.90-1.50 (PVA CH₂), 1.36-1.48 (NB CH₂).

### Synthesis of PVA-Norb

The reaction to obtain a suitable anhydride compound as modifying agent was carried out under Argon atmosphere. 5-Norbornene-2-carboxylic acid (5.02 g, 36.3 mmol) was dissolved in 20 mL of dry dichloromethane (DCM). A suspension of EDC·HCl (3.48 g, 18.2 mmol) in 20 mL of DCM was added to the solution of the carboxylic acid and the resulting solution was stirred at room temperature for 3 h.

Then the reaction mixture was diluted with DCM to a total of 60 mL and washed two times with 50 mL of deionized water and two times with 5 wt % aqueous sodium bicarbonate solution. The washed DCM phase was dried over sodium sulfate and solvent was removed using a rotary evaporator to yield the corresponding carboxylic acid anhydride of 5-norbornene-2-carboxylic acid as an off-white, solid product (53% yield). 1H-NMR (400 MHz, D₂O): 6.10 (CH═CH), 3.23 (CH), 3.08 (CH), 2.94 (CH), 1.92 (CH2), 1.45 (CH₂), 1.27 (CH2).

The subsequent modification of PVA with norbornene groups was carried out under Argon atmosphere. PVA (5 g, 27 kDa, 98% degree of hydrolysis) was dissolved in 40 mL of DMSO (dimethyl sulfoxide, anhydrous) at 70 °C. Temperature was then decreased and kept at 60 °C. The modification reaction was started by adding a solution of 5-norbornene-2-carboxylic anhydride (2.42 g, 9.35 mmol) in 20 mL DMSO to the stirred PVA solution. The obtained solution was stirred for 20 h at 60 °C under Argon atmosphere. The product-containing solution was then dialyzed against 10 mM sodium bicarbonate solution and deionized water with multiple changes for at least 48 h. Lyophilization of the dialyzed solution yields the polymer as off-white solid (~75% yield). The degree of substitution was determined via proton NMR spectroscopy: About 5% of repeating units of PVA were modified with norbornene groups. 1H-NMR (400 MHz, D₂O): 6.37-5.90 (Norb CH═CH), 4.05 (PVA CH), 3.30-2.73 (Norb CH), 2.13 (PVA-Acetyl CH₃), 1.71 (PVA CH₂), 1.40 (Norb CH₂).

### Hydrogel Formation

Hydrogel formulation preparation steps were carried out under light protection to prevent premature reaction. To generate a photocrosslinkable formulation of PVA via a thiol-ene mechanism, the PVA modification (e.g. modified with norbornene groups, PVA-NB) and the multifunctional thiol component were dissolved separately in a photoinitiator stock solution (e.g. Irgacure 2959, α-hydroxy-4-(2-hydroxyethoxy)-α-methylpropiophenon in deionized water or PBS (pH=7.4, e.g. Lonza) or mixtures with ethanol to allow solubility of alternative crosslinkers) and mixed to achieve a specific concentration of network-forming agents (e.g. 10 wt % as sum of PVA-NB and multifunctional thiol) and molar ratio of thiol groups and ene groups (e.g. a molar thiol-ene ratio of 0.5, i.e. 50 mol %). Before photocrosslinking, the formulation was mixed thoroughly. Formulation was pipetted into a silicone mold for UV crosslinking. Crosslinking can be performed preferably in suitable UV irradiation settings (e.g. INTELLI RAY 600 from Uvitron International, or Omnicure S2000 lamp). Crosslinking using other suitable strategies is also possible and may include alternative initiator systems (e.g. azo compounds, persulfates or other peroxides, metal ions).

### Particle Production

Hydrogels were placed in porcelain dishes and swollen in deionized water for 3.5 h. After that, the water was removed with the help of a Büchner funnel. Ethanol was added for a deswelling step. This was carried out for 24 h with ethanol being changed once. Gels were dried at 60 °C in a vacuum oven at 200-300 mbar and on a rotary evaporator at 5 mbar until weight constancy was achieved.

Dried material was first ground manually with pestle and mortar and liquid nitrogen cooling. The pre comminuted material was milled using suitable milling equipment (e.g. a water-cooled laboratory mill (IKA A10 basic with steel cutters), or a Retsch centrifugal mill (ZM200). Subsequently, particle size fractions were generated by sieving (steel mesh sieves of appropriate mesh size (Retsch).

### Particle Production Process

### Hydrogel Synthesis and Matrix Preparation

Photoinitiator (PI) stock solutions were prepared in PBS (pH=7.4, Lonza) and in PBS/EtOH (1:2). Irgacure2959 (“I2959”, α-hydroxy-4-(2-hydroxyethoxy)-α-methylpropiophenon, Ciba) was dissolved to reach a concentration of 0.25% (wt/vol) in both solvents. All following hydrogel formulation preparation steps including the PI stock were carried out under light protection to prevent premature reaction.

Generalized procedures are described to produce hydrogel materials and more detailed information about each respective formulation can be seen in Tables 4 and 5: To produce hydrogels crosslinked with DTT, the PI stock solution in PBS was used. To produce hydrogels crosslinked with GDMA, the PI stock solution in PBS/EtOH was used. PVA macromer was dissolved in PI stock solution. To facilitate dissolution of the macromer the mixture was heated to ~50 °C and agitated frequently. Appropriate amounts of dithiol crosslinker were weighed into a tube and also dissolved in PI stock solution.

Finally, the PVA macromer solution and the thiol crosslinker solution were mixed well to achieve liquid hydrogel formulations of ~10 wt % of network forming components (i.e. thiol crosslinker+PVA macromer). The final formulation was poured into Petri dishes and irradiated for 30 s for crosslinking in a UV chamber (Uvitron International IntelliRay 60, set to 50% intensity). Crosslinked hydrogels were carefully removed from the molds using a wetted spatula and hydrogels were immersed in an excess of PBS for at least 24 h with one medium change to remove unreacted material and PI. Washed hydrogels were immersed in distilled ethanol for at least 48 h with two changes to partially dehydrate the polymer networks. Partially dehydrated networks were dried in a vacuum oven (200-300 mbar, 50 °C).

### Material Characterization

### Swelling Properties

### Volumetric Swelling

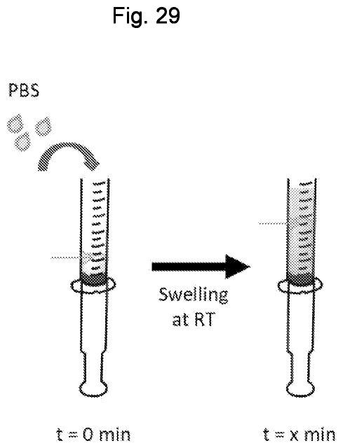

For volumetric swelling, particles were packed loosely into a cylindrical container with graduation markings. The particle volume was noted of the dry particles using the graduation marks of the container. The experimental setup is also shown in FIG. 29.

An excess of PBS was added without agitating the particles to start the swelling procedure. The volume of the particle phase during swelling in PBS was noted at certain time points (0, 0.5, 1, 1.5, 2, 2.5, 3.5, 4, 4.5, 5, 6, 7, 8, 9, 10, 12, 14, 16, 18, 20, 60 min, and 24, and 48 h) and the relative volume increase was calculated as percentage according to the following equation. Experiments were conducted as triplets or quadruplets (n=3, n=4) and mean and standard deviation were calculated.

$$Relative Volume Increase in \% = \frac{[ Volume after swelling ] - [ Volume dry ]}{[ Volume dry ]} \cdot 100$$

### Mass Swelling (Gravimetric Swelling)

To quantify gravimetric swelling of the particle matrices, particles (100-150 mg) were filled into polypropylene vials. At the start of the experiments the dry weight of the particles was determined. The particles were then mixed with an excess of PBS (pH=7.4) and vortexed for 3-5 seconds to ensure complete wetting and swelling of the materials. After 24 h of swelling the supernatant PBS was removed (with optional centrifugation) and the swollen particle mass was recorded.

The mass swelling ratio was calculated according to the following equation. Experiments were conducted as triplicates (n=3) and mean and standard deviation were calculated.

$$Water Uptake in \% = \frac{[ Weight after swelling ] - [ Weight dry ]}{[ Weight dry ]} \cdot 100$$

1: A hemostatic product suitable for administration to a human patient, the hemostatic product comprising a biocompatible hemostatic material comprising non-porous, cross-linked polyvinyl alcohol particles (PVA particles).

2: The hemostatic product according to claim 1, wherein at least 50% of the PVA particles have a diameter of less than 500 μm.

3: The hemostatic product according to claim 1, wherein a ratio of PVA particles with a diameter of less than 200 μm to PVA particles with a diameter of between 200 and 500 μm is from 10:90% (w/w) to 90:10% (w/w).

4: The hemostatic product according to claim 1, wherein the PVA particles are absorbable to more than 90% by the body of the human patient within 6 months after administration to the human patient.

5: The hemostatic product according to claim 1, wherein the PVA particles are non-porous, which is defined as having a solid volume of at least 70% as determined by a nano-CT measurement.

6: The hemostatic product according to claim 1, wherein the PVA particles are free of covalently or non-covalently bound blood compounds of natural or recombinant origin, or functional fragments of such blood compounds.

7: The hemostatic product according to claim 1, wherein the product is free of bound blood compounds of natural or recombinant origin, or functional fragments of such blood compounds.

8: The hemostatic product according to claim 1, wherein the PVA particles have a substitution degree of 2% to 70%.

9: The hemostatic product according to claim 1, wherein the PVA particles have a substitution degree of 1% or more.

10: The hemostatic product according to claim 1, wherein the PVA particles are non-porous, cross-linked norbornene-group-modified particles (PVA-NB particles).

11: The hemostatic product according to claim 10, wherein the PVA-NB particles were formed by crosslinking by thiol groups.

12: The hemostatic product according to claim 1, wherein the product contains 20 to 70% of PVA particles with a size of 100 to 200 μm and 10 to 60% of PVA particles with a size of 200 to 500 μm.

13: The hemostatic product according to claim 1, wherein the PVA particles have a swelling capacity of at least 100% relative volume increase after 60 minutes.

14: The hemostatic product according to claim 1, wherein the product and/or the PVA particles is/are aseptic by γ-radiation sterilization treatment.

15: The hemostatic product according to claim 1, wherein the product is sterile.

16: The hemostatic product according to claim 1, wherein the product does not comprise an additional coagulatively active agent further to the PVA particles.

17: The hemostatic product according to claim 1, wherein the product is part of a composite hemostatic product or hemostatic kit.

18: The hemostatic product according to claim 1, wherein the PVA has a molecular weight of 3 to 100 kDa.

19: The hemostatic product according to claim 1, for human or animal therapy and/or surgery.

20: A method for treatment of a wound or for achieving hemostasis in a human patient, the method comprising administering the hemostatic product according to claim 1 to a patient with a wound or in need of hemostasis.

21: A method for producing a hemostatic product comprising cross-linked non-porous PVA particles, wherein polyvinyl alcohol is modified with a modifying agent comprising at least one of an modifying agent comprising an “ene” and/or “yne” group; and cross-linked to form PVA particles; without applying a step causing a porous structure to be formed.

22: The method according to claim 21, wherein the polyvinyl alcohol used as a starting material has a number of residual acetyl groups of 5% or less.

23: The method according to claim 21, wherein the cross-linked polyvinyl alcohol is milled to obtain cross-linked polyvinyl alcohol particles.

24: The method according to claim 21, wherein the polyvinyl alcohol is chemically modified.

25: The method according to claim 21, wherein the product and/or the PVA particles is sterilized.

26: The method according to claim 21, wherein polyvinyl alcohol is modified with norbornene groups to obtain a modified polyvinyl alcohol, and the modified polyvinyl alcohol is cross-linked by UV-initiated thiol-ene chemistry.

27: The method according to claim 21, wherein the PVA particles are milled.

28: The method according to claim 21, wherein the PVA has a molecular weight of 3 to 100 kDa.

29: A non-porous, biocompatible hemostatic material comprising cross-linked polyvinyl alcohol, obtainable by the method according to claim 21.

30: A kit comprising the hemostatic product according to claim 1 and further comprising at least one administration device, buffer solution, syringe, tube, catheter, forceps, scissors, sterilizing pad or lotion.

31: The kit according to claim 30, wherein the buffer solution further comprises a component selected from the group consisting of an anti-bacterial agent, a coagulatively active agent, an immunosuppressive agent, an anti-inflammatory agent, an analgesic agent, an anti-fibrinolytic agent, a growth factor, a vitamin, a cell, and mixtures thereof.

32: The kit according to claim 30, further comprising a dry preparation of a further active agent.
